(19) United States
(12) Patent Application Publication
(10) Pub. No.: US 2024/0050526 A1
(43) Pub. Date: Feb. 15, 2024

(54) CATH2 DERIVATIVES FOR STIMULATING INNATE IMMUNE MEMORY

(71) Applicant: Universiteit Utrecht Holding B.V., Utrecht (NL)

(72) Inventors: Hendrik Peter HAAGSMAN, Utrecht (NL); Albert VAN DIJK, Utrecht (NL); Edwin Johannes Adrianus VELDHUIZEN, Utrecht (NL)

(21) Appl. No.: 18/267,693

(22) PCT Filed: Dec. 17, 2021
(86) PCT No.: PCT/NL2021/050776

§ 371 Date: Jun. 15, 2023

(30) Foreign Application Priority Data
Dec. 18, 2020 (EP) 20215665.9

(51) Int. Cl. A61K 38/17; A61K 39/39; A61P 37/04

(52) U.S. Cl. CPC A61K 38/1729; A61K 39/39; A61P 37/04; A61K 2039/55516

(57) ABSTRACT

The invention relates to methods for activating, inducing or promoting innate immune memory in a subject in need thereof comprising administering to the subject CATH2 or a derivative thereof. The invention further relates to methods of improving antimicrobial treatment in a subject in need thereof comprising administering to the subject CATH2 or a derivative thereof and to a use of CATH2 or a derivative thereof as an adjuvant for a pathogen-specific vaccine.

### FIELD OF THE INVENTION

The invention relates to the field of medical and veterinary science, in particular to peptides with immune stimulating activity, especially CATH2 derivatives.

### BACKGROUND OF THE INVENTION

Innate immune memory or “trained immunity” has been demonstrated to be induced by various microbial components in NK cells and monocytes/macrophages [1-4]. Bacterial, fungal and viral ligands can reprogram the monocyte phenotype via activation of pattern recognition receptors (PRR) towards an enhanced (trained) or diminished (tolerance) immune response to re-stimulation. Previous exposure to heat-killed Candida albicans or the fungal cell wall component ß-glucan was shown to generate protection against re-infection in mice in a macrophage dependent manner, through an amplification of pro-inflammatory cytokine response to TLR2 and TLR4 ligands [2]. In addition to ß-glucans, in vitro trained immunity can be elicited in monocytes/macrophages by components of Gram-positive bacteria (muranyl dipeptide) [2, 3], BCG (Bacille Calmette-Guarin) [3], low doses of polysaccharides [1] and oxidized low-density lipoprotein [5]. The mechanisms underlying trained immunity are an altered cell metabolism towards aerobic glycolysis and changes in the epigenetic landscape at specific loci containing immune-related genes [6].

Cathelicidins are host defence peptides (HDPs) and part of the innate immune system [7]. These peptides are known endogenous alarmins that are passively (necrosis) or actively released through microbial exposure or neutrophil and mast cell degranulation upon tissue injury or infection [8]. Potent immunomodulatory effects on macrophages have been reported for human cathelicidin LL-37 and chicken CATH-2 in vitro [9-12]. In vivo, antimicrobial efficacy of cathelicidin-derived peptides was demonstrated in mouse infection models for invasive Staphylococcus aureus [13, 14], MRSA [15], Escherichia coli [13] and Mycobacterium tuberculosis [14] infection. To increase the therapeutic potential of cathelicidin-derived peptides, a full D-amino acid analog can be used to gain high resistance against proteases while maintaining low immunogenicity [16]. Prophylactic treatment of chicken embryos by in ovo injection with DCATH-2 considerably reduced colibacilossis-associated mortality and morbidity [17]. In addition, delayed mortality was observed when DCATH-2 was injected into the yolk of zebrafish embryos followed by intravenously infection with a lethal dose of Salmonella enterica [18].

Although several antimicrobial treatments are known, there remains a need in the art for improved methods of treatment and prevention of in particular infectious disease.

### SUMMARY OF THE INVENTION

It is an object of the present invention to provide novel uses of CATH2 and derivatives thereof. The invention therefore provides a method for activating, inducing or promoting innate immune memory in a subject in need thereof comprising administering to the subject CATH2 or a derivative thereof.

In a further aspect, the invention provides CATH2 or a derivative thereof for use in a method for activating, inducing or promoting innate immune memory in a subject in need thereof.

In a further aspect, the invention provides a use of CATH2 or a derivative thereof in the preparation of a medicament for activating, inducing or promoting innate immune memory in a subject in need thereof.

In a further aspect, the invention provides a method for activating or inducing an innate immune memory in a subject in need thereof, the method comprising administering to said subject an effective amount of CATH2 or a derivative thereof, thereby activating or inducing innate immune memory in said subject.

In a further aspect, the invention provides CATH2 or a derivative thereof for use in a method for activating or inducing an innate immune memory in a subject in need thereof.

In a further aspect, the invention provides a method for treating an inactivated or defective innate immune memory in a subject, the method comprising administering to said subject an effective amount of CATH2 or a derivative thereof, thereby treating said inactivated or defective innate immune memory in said subject.

In a further aspect, the invention provides CATH2 or a derivative thereof for use in a method for treating an inactivated or defective innate immune memory in a subject, in particular a subject in need thereof.

In a further aspect, the invention provides a method for improving or enhancing antimicrobial activity of an antimicrobial agent. In a preferred embodiment, the treatment is treatment of Salmonella enteritidis, Candida albicans or Escherichia coli, more preferably treatment of Salmonella enteritidis or Candida albicans.

In a further aspect, the invention provides CATH2 or a derivative thereof for use in a method for improving or enhancing antimicrobial activity of an antimicrobial agent.

In a further aspect, the invention provides a use of CATH2 or a derivative thereof in the preparation of a medicament for improving or enhancing antimicrobial activity of an antimicrobial agent.

In a further aspect, the invention provides a use of CATH2 or a derivative thereof as an adjuvant for a pathogen-specific vaccine.

In a further aspect, the invention provides CATH2 or a derivative thereof for use as an adjuvant for a pathogen-specific vaccine, preferably for use as an adjuvant for a pathogen-specific vaccine in a method for the treatment or prevention of an infectious disease caused by said pathogen. Preferably, a composition comprising the CATH2 or a derivative thereof and the pathogen-specific vaccine for use in a method for the treatment or prevention of an infectious disease caused by said pathogen.

In a further aspect, the invention provides a use of CATH2 or a derivative thereof in the preparation of an adjuvant for a pathogen-specific vaccine, preferably in the preparation of a medicament comprising the CATH2 or derivative thereof and the pathogen-specific vaccine, preferably for the treatment or prevention of an infectious disease caused by said pathogen.

In a further aspect, the invention provides a method for the treatment or prevention of an infectious disease caused by a pathogen, comprising administering a pathogen-specific vaccine that is specific for said pathogen and CATH2 or a derivative thereof as an adjuvant, preferably wherein the pathogen-specific vaccine is an (attenuated) pathogen or pathogen derived peptide or protein.

### DETAILED DESCRIPTION

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 10% of the value.

In one embodiment, the methods and uses of the invention are for the treatment of existing disease, in particular infectious disease, preferably of bacterial infection, such as E. coli or Salmonella, in particular in particular S. enteritidis, infection, or a fungal infection, such as Candida albicans infection. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In one embodiment, the methods and uses of the invention are for prevention of disease, in particular infectious disease, preferably of bacterial infection, such as E. coli or Salmonella, in particular in particular S. enteritidis, infection, or a fungal infection, such as Candida albicans infection. As used herein, the term “prevention” refers to precluding or delaying the onset of a disease or condition and/or the appearance of clinical symptoms of the disease or condition in a subject that does not yet experience clinical symptoms of the disease.

The term “peptide” as used herein means a sequence of amino acids that are coupled by peptide bonds, wherein the amino acids are one of the twenty naturally peptide-building amino acids and wherein one or all of the amino acids can be in the L-configuration or in the D-configuration, or, for isoleucine and threonine in the D-allo configuration (only inversion at one of the chiral centers). A peptide according to the invention can be linear, i.e. wherein the first and last amino acids of the sequence have a free NH2- or COOH-group respectively or are N-terminally (acetylation) and/or C-terminally (amidation) modified. In amino acid sequences as defined herein amino acids are denoted by single-letter symbols or three-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Lu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

The terms “Innate immune memory” and “trained immunity” are used interchangeably herein and refer to the ability of innate immune cells to functionally reprogram after exogenous or endogenous insults and to respond non-specifically to a subsequent challenge after return to a non-activated state. Trained immunity is orchestrated by epigenetic modifications leading to changes in gene expression and cell physiology of the innate immune cells. The innate immune memory provides a powerful tool to regulate the delicate balance of immune homeostasis, priming, training and tolerance of innate immune cells. The long-term adaptation demonstrated with trained immunity can be used to achieve long-term therapeutic benefits with a more strongly response in a range of immune-related diseases, including infectious disease, as compared to direct treatment with antimicrobial agents.

The present inventors found that CATH2 and derivatives thereof are potent stimulators of innate immune memory. As shown in the examples herein, it was found that CATH2 and derivatives induce trained immunity in innate immune cells (macrophages) leading to enhanced proinflammatory cytokine production upon repeated stimulation with different TLR ligands. In particular, it was shown that:
1) Restimulation of CATH2 trained, phorbol 12-myristate 13-acetate (PMA) differentiated, dTHP-1 cells with LPS (TLR4) amplifies their proinflammatory cytokine response (IL-6). THP1 cells are monocyte-like (non-adherend) cells, PMA-differentiation generates a macrophage-like phenotype (adherend).
2) Restimulation of DCATH2 trained dTHP-1 cells with Pam3CSK4 (TLR1/2), Pam2CSK4 (TLR2/6), smooth or rough LPS (TLR4) amplifies their proinflammatory cytokine response (TNF-alpha, IL-6).
3) L-, DCATH2 training does not alter basal dTHP1 cell TNF-alpha and IL-6 production.
4) DCATH2 trained dTHP-1 cells have enhanced antimicrobial activity against Salmonella enteritidis and Candida albicans.
5) DCATH2 training shifts dTHP-1 cellular metabolism towards mTOR-dependent aerobic glycolysis and long-chain fatty acid accumulation, which are both maintained during LPS stimulation.
6) DCATH2 training of dTHP-1 cells is dependent on epigenetic regulation (histone acetylation).
7) DCATH2 training of dTHP-1 cells is dependent on MAPK p38 signal transduction.
8) DCATH2 training of THP-1 cells is receptor mediated, and dependent on purinergic signalling by P2 family receptors in general and specifically P2X7R, a key regulator of aerobic glycolysis and involved in peptide internalization.

All these findings taken together demonstrate that CATH2 and derivatives are able to activate, induce and stimulate trained immunity.

In a first aspect, the invention thereof provides a method for activating, inducing or promoting innate immune memory in a subject in need thereof comprising administering to the subject CATH2 or a derivative thereof. Also provided is CATH2 or a derivative thereof for use in a method for activating, inducing or promoting innate immune memory in a subject in need thereof.

As used herein, activating, inducing or stimulating the innate immune memory means that the innate immune memory is activated, induced or stimulated after administration of CATH2 or derivative thereof as compared to prior to administration of the CATH2 or derivative thereof. This activation, induction or stimulation can in particular be determined after restimulation following administration of the CATH2 or derivative thereof, as basal features, such as TNF-alpha and IL-6 production, mTOR-dependent aerobic glycolysis and long-chain fatty acid accumulation, of immune cells have been demonstrated not to be affected after administration of CATH2 or a derivative. For instance as described in the examples herein restimulation with TLR4 ligands and measuring cytokine production levels, such as TNF-alpha and/or IL-6, biased cellular metabolism towards mTOR-dependent aerobic glycolysis and long-chain fatty acid accumulation.

In one embodiment, the CATH2 and derivatives induce trained immunity in innate immune cells. Innate immune cells are white blood cells that mediate innate immunity and include basophils, dendritic cells, eosinophils, Langerhans cells, mast cells, monocytes and macrophages, neutrophils and NK cells.

In one embodiment, the innate immune memory is innate immune memory for infectious disease, i.e. bacterial, viral, fungal or parasitic infection. The infectious disease is in particular a bacterial infection by both Gram-positive and Gram-negative bacteria, or a fungal infection, such as Candida albicans infection. In one embodiment, the treatment or prevention of infectious disease, in particular bacterial infections by both Gram-positive and Gram-negative bacteria. Infectious disease and bacterial infections that can be suitably treated and/or prevented in accordance with the present inventions are described in more detail herein below.

In one embodiment, the method is for improving or enhancing antimicrobial treatment with an antimicrobial agent, in particular the CATH2 or derivative thereof. In a further aspect, provided is a method of improving antimicrobial treatment in a subject in need thereof comprising administering to the subject CATH2 or a derivative thereof. Also provides is CATH2 or a derivative thereof for use in a method of improving antimicrobial treatment in a subject in need thereof. Now that the present inventors have identified that CATH2, DCATH2 and derivatives thereof induce innate immune memory, it has become possible to improve existing antimicrobial therapies using these, thereby improving treatment outcomes and/or treatment efficiency. In particular, the improvement is an improvement in treatment efficiency, such as in timing of administration of the CATH2 or derivative, dosing of the CATH2 or derivative, formulation of the CATH2 or derivative and/or administration routes, combination with other active or non-active compounds as described elsewhere in more detail. In particular, now that is known that CATH2 and derivatives stimulate innate immune memory, it has become possible to select an appropriate formulation, including appropriate auxiliaries, such as adjuvants, and/or combination with other active or non-active ingredients and administrations dosages and schemes that support the activation, induction or promotion or innate immune memory.

In one embodiment, the subject to be treated or administered CATH2 or a derivative thereof in accordance with the invention is a subject in need of activating, inducing or stimulating an innate immune memory.

Animals are exposed to numerous potential pathogens daily, through contact, ingestion, and inhalation. The ability to avoid infection depends in part on the adaptive immune system, which remembers previous encounters with specific pathogens and destroys them when they attack again. However, the adaptive immune responses are slow to develop on first exposure to a new pathogen, as specific clones of B and T cells have to become activated and expand. It can therefore take at least a week before the responses are effective. By contrast, a single bacterium with a doubling time of one hour can produce millions of progenies, a full-blown infection, in a single day. Therefore, during the first critical hours and days of exposure to a new pathogen, the animals rely on their innate immune system to protect them from infection. Innate immune responses are not specific to a particular pathogen in the way that the adaptive immune responses are. They depend on a group of proteins, phagocytic cells (e.g., monocytes, macrophages, neutrophils, dendritic cells, and mast cells), and non-phagocytic cells (e.g., NK cells) that recognize conserved features of pathogens and become quickly activated to help destroy invaders. Therefore, the quick activation of innate immune system is critical for animals. Some animals have the problem of an inability to induce or activate their innate immune memory. This could be due to a mechanistic disorder in a group of proteins and phagocytic cells that recognize conserved features of pathogens. These animals have an inactivated or defective innate immune memory. They are in need of an activated or induced innate immune memory in order to provide an immunity protection against a pathogen, a disease or a condition.

Whether or not a subject is in need of an activated, induced or stimulated innate immune memory can be determined based on the knowledge in the art and using methods known in the art to the skilled person. In particular, as described above, a subject that has an inability to induce or activate its innate immune memory, due to a disorder or defect in a group of proteins, phagocytic cells or non-phagocytic cells (NK cells) that recognize conserved features of pathogens, is a subject in need of an activated, induced or stimulated innate immune memory. Hence, one possibility is to assess those proteins and/or phagocytic cells or NK cells, in particular at phagocytic cell or NK cell behaviour to determine whether a subject has an inability to induce or activate its innate immune memory and thus is in need of an activated, induced or stimulated innate immune memory.

In some embodiments a method or use of the invention comprises determining whether a subject is in need of an activated, induced or stimulated innate immune memory. In preferred embodiments, said determining comprises determining functionality of phagocytic cells (e.g. phagocytic activity), such as monocytes, neutrophils, basophils and mast cells, or functionality of natural killer cells, for instance after stimulation to bacterial products, In some embodiments, said determining comprises determining or measuring cytokine production (e.g. TNF-alpha, IL-1, IL-12, interferon α, interferon γ and/or IL-6) after stimulation by e.g. bacterial products. Said determination if preferably performed in vitro using cells isolated from the subject, e.g. isolated from blood of the subject.

The invention further provides a method for treating an inactivated or defective innate immune memory in a subject, in particular in a subject in need thereof, the method comprising administering to said subject an effective amount of CATH2 or a derivative thereof, thereby treating said inactivated innate immune memory in said subject. Also provided is CATH2 or a derivative thereof for use in a method for treating an inactivated or defective innate immune memory in a subject. in particular a subject in need thereof. As used herein “inactivated innate immune memory” and “defective innate immune memory” refer to a condition that is characterized by an inability or reduced ability to induce or activate its innate immune memory, in particular due to a mechanistic disorder in a group of proteins and phagocytic cells that recognize conserved features of pathogens. Whether or not a subject is suffering from an inactivated or defective innate immune memory can be determined as described herein above for determining whether a subject is in need of an activated, induced or stimulated innate immune memory, using methods known in the art.

As used herein, the terms “CATH2” and “CMAP27” are used interchangeably. Like other members of the cathelicidin family CMAP27 is encoded as a prepropeptide (154 amino acids) and after proteolytic processing, a C-terminal peptide is released that has demonstrated potent broad spectrum antimicrobial activity. The 27 amino acid sequence of this C-terminal peptide, called CMAP27 or CATH2, is RFGRFLRKIRRFRPKVTITIQGSARFG. As used herein, a “CATH2 derivative” generally refers to a peptide that is a derivative of CATH2 in that it contains at least part of the sequence of CATH2 and that has maintained at least one antimicrobial properties of CATH2, although not necessarily to the same extent. In particular, antimicrobial activity against Gram(−) bacteria is maintained.

In one preferred embodiment, the CATH2 derivative is selected from the group consisting of C-terminally and/or N-terminally truncated CATH2 derivatives, D-amino acid CATH2 derivatives, C-terminally or N-terminally truncated D-amino acid CATH2 derivatives, cyclic CATH2 derivatives and inverso and retroinverso CATH2-derivatives. The derivative may contain one or more amino acid substitutions, preferably 1 to 3 amino acid substitutions, more preferably 1 or 2 amino acid substitutions. Preferably, the CATH2 derivative is selected from the group consisting of C-terminally and/or N-terminally truncated CATH2 derivatives, D-amino acid CATH2 derivatives and C-terminally or N-terminally truncated D-amino acid CATH2 derivatives, such as C-terminally or N-terminally truncated DCATH2. In one preferred embodiment, CATH2 or DCATH2 is used. DCATH2 is the full length CATH2 peptide consisting of D-amino acids.

“C-terminally truncated CATH2 derivatives” refers to truncated peptides lacking one or more amino acids at the C-terminus of CATH2, preferably lacking up to 17 amino acids, more preferably up to 12 amino acids, more preferably up to 6 amino acids. Preferred examples are described in WO 2010/093245, which is incorporated herein by reference, and especially the peptides listed as CMAP26-NH₂, CMAP26, CMAP26 (P14→G), CMAP26 (P14→L), CMAP1-21, CMAP1-15, CMAP1-15 (F2→L), CMAP1-15 (F5→L), CMAP1-15 (F12→L), CMAP1-15 (3xF→L), CMAP1-15 (F2→W), CMAP1-15 (F5→W), CMAP1-15 (F12→W), CMAP1-15 (F2→W; F5→W; F12→W), CMAP1-13, CMAP1-12, CMAP1-11 and CMAP1-10 in Table 1 of said document and their acetylated and/or amidated derivatives are preferred. Herein, and in all amino acid sequence defined herein, the arrow notation indicates an amino acid substitution. For instance, F2→L indicates that the F at position 2 is replaced by L and F2, 5→W indicates that F at positions 2 and 5 is replaced by W. Further preferred are CMAP1-21 (F2→W), CMAP1-21 (F5→W), CMAP1-21 (F12→W), CMAP1-21 (F2, 5→W), CMAP1-21 (F5, 12→W), CMAP1-21 (F2, 12→W), CMAP1-21 (F2, 5, 12→W), CMAP1-21 (F2→Y), CMAP1-21 (F5→Y), CMAP1-21 (F12→Y), CMAP1-21 (F2, 5→Y), CMAP1-21 (F5, 12→Y), CMAP1-21 (F2, 12→Y), CMAP1-21 (F2, 5, 12→Y), CMAP1-21 (F2→W; F5→Y), CMAP1-21 (F2→Y; F5→W), CMAP1-21 (F5→W; F12→Y), CMAP1-21 (F5→Y; F12→W), CMAP1-21 (F2→W; F12→Y), CMAP1-21 (F2→Y; F12→W), CMAP1-21 (F2→W; F5→Y; F12→Y), CMAP1-21 (F2→Y; F5→W; F12→Y), and CMAP1-21 (F2→Y; F12→Y; F12→W). Preferred examples of C-terminally truncated CATH2 derivatives are also described in WO2015/170984, which is incorporated herein by reference. The CMAP proteins identified above, may also be indicates as CATH2 peptides. CMAP1-21 then would be CATH2(1-21).

“N-terminally truncated CATH2 derivatives” are CATH2 derivatives that are truncated at the N-terminal amino acid (arginine) of CATH2 thus lacking one or more amino acids at the N-terminus of CATH2, preferably lacking up to 10 amino acids, more preferably up to 7 amino acids, more preferably up to 6 amino acids. Preferred are the derivatives selected from the group consisting of N-terminally truncated variants of CMAP1-21: CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

“D-amino acid CATH2 derivatives” are CATH2 derivatives as defined herein (including the above defined C- and N-terminally truncated CMAP27-derivatives) that contain at least one amino acid in the D configuration. A special category of these D-amino acid CATH2 derivatives are the peptides that are composed of only D amino acids (i.e. in which no L amino acid is present). This special category is herein defined as DCATH2. Also CATH2 itself, comprising one or more, or, alternatively, all D amino acids is comprised within this definition. Preferred D-amino acid CATH2 derivatives are DCATH2 and the following D-amino acid CATH2 derivatives (where all amino acids are in the D-form):

| | |
|---|---|
| D-C (1-26) | RFGRFLRKIRRFRPKVTITIQGSARF-NH₂ |
| D-C (1-21) | REGRELRKIRRFRPKVTITIQ-NH₂ |
| D-C (4-21) | RFLRKIRRFRPKVTITIQ-NH₂ |
| D-C (7-21) | RKIRRFRPKVTITIQ-NH₂ |
| D-C (7-21) F/W | RKIRRWRPKVTITIQ-NH₂ |
| D-C (7-21) F/Y | RKIRRYRPKVTITIQ-NH₂ |
| D-C (10-21) F/W | RRWRPKVTITIQ-NH₂ |
| D-C (1-15) | REGRELRKIRRFRPK-OH |

Particularly preferred DCATH2 derivatives are DC(1-21) and DC(4-21), in particular DC(1-21).

“Cyclic CATH2-derivatives” are CATH2 derivatives in which at least two non-adjacent amino acids are connected to form a ring structure. Although in principle any chemical binding construction may be used, such as replacing two non-adjacent amino acids in any of the above-mentioned CATH2 derivatives with a cysteine, where these cysteines then form an S-S bridge, a preferred binding system uses the binding between Bpg (Fmoc-L-bishomopropargylglycine) and an azido-resin, wherein the Bpg is attached to an internal arginine, leucine, phenylalanine or tryptophane residue and the azido-resin is attached to the C-terminal glutamic acid residue. Especially, such cyclic derivatives are:

| | |
|---|---|
| cycCMAP(1-21)[Lys8] | RFGRFLR(Bpg)IRRFRPKVTITIQ(azido-resin) |
| cycCMAP(1-21)[Arg7] | RFGRFL(Bpg)KIRRFRPKVTITIQ(azido-resin) |
| cycCMAP(1-21)[Leu6] | RFGRF(Bpg)RKIRRFRPKVTITIQ(azido-resin) |
| cycCMAP(1-21)[Leu6], Phe2/Trp | RWGRF(Bpg)RKIRRFRPKVTITIQ(azido-resin) |
| cycCMAP(1-21)[Leu6], Phe2, 5/Trp | RWGRW(Bpg)RKIRRFRPKVTITIQ(azido-resin) |
| cycCMAP(1-21)[Leu6], Phe2, 5, 12/Trp | RWGRW(Bpg)RKIRRWRPKVTITIQ(azido-resin) |
| cycCMAP(1-21)[Leu6], Phe5, 12/Trp | RFGRW(Bpg)RKIRRWRPKVTITIQ(azido-resin) |
| cycCMAP(1-21)[Leu6], Phe12/Trp | RFGRF(Bpg)RKIRRWRPKVTITIQ(azido-resin) |

“Inverso” and “Retroinverso” CATH2 derivatives (“I”-CATH2 and “RI”-CATH2 derivatives) are peptides that have an inverted sequence with respect to the above-mentioned CATH2 derivatives, in the sense that the amino acids are connected to each other in a reverse order. When the inverted CATH2 derivatives contain one or more D amino acids they are termed “Retroinverso” or “RI”. If the inverted derivative only contains L-amino acids it is termed “Inverso” or “I”. The I and RI equivalent of CATH2 then become GFRASGQITITVKPRFRRIKRLFRGFR and other preferred examples of such I or RI-CMAP27-derivatives are:

| | |
|---|---|
| RI-C (1-21) | QITITVKPRFRRIKRLERGER |
| RI-C (4-21) | QITITVKPRERRIKRLER |
| RI-C (7-21) | QITITVKPRFRRIKR |
| RI-C (7-21) F/W | QITITVKPRWRRIKR |
| RI-C (7-21) F/Y | QITITVKPRYRRIKR |
| RI-C (10-21) F/W | QITITVKPRWRR |

The I and RI-CMAP27 derivatives may be acetylated at their N-terminal and/or amidated at their C-terminal.

In a preferred embodiment, the CATH2 or derivative thereof used in any method or use of the invention is CATH2, DCATH-2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y), more preferably wherein the CATH2 or derivative is CATH2, DCATH2, DCATH2(1-21) or DCATH2(4-21). In one embodiment, the CATH2 or derivative thereof used in any method or use of the invention is DCATH-2, DCATH2(1-21) or DCATH2(4-21).

As used herein, the term “subject” encompasses humans and animals, including livestock and farm animals such as dairy cattle and beef cattle, including cows and buffaloes, sheep, goats, alpacas, horses, mules, donkeys, camels, llamas, pigs, fish, rodents and poultry, dogs, cats, chinchillas, ferrets, birds, hamsters, rabbits, mice, gerbils, rats, and guinea pigs. In one preferred embodiment, the subject is a mammal, such as mammalian farm animals, livestock or pets. In one preferred embodiment, the subject is an avian subject, more preferably poultry. The term poultry includes chicken, ducks, goose, pheasants and turkeys. In a preferred embodiment, the subject is chicken or turkey, more preferably chicken.

Inducing or improving innate immune memory and/or improving antimicrobial treatment is in particular advantageous in subjects in having a weakened innate immune system or otherwise in need of enhanced innate immune memory. Therefore, in one embodiment, the subject in need thereof is a subject in need of enhanced innate immune memory or a subject having a weakened innate immune memory. Examples of such subjects are subjects suffering from infection, such as bacterial, viral or fungal infection, or stressed subjects. Other examples are subjects suffering from dysregulated activation or inappropriate induction of trained immunity, which can lead to immunodepression. The methods of the present invention may help to restore appropriate innate immune memory.

Particularly useful is the application of CATH2 or a derivative thereof in farms or stables where animals are kept together, such as in farming, including poultry, cattle, pig, goat and sheep farming. Infectious disease occurring in such environments may quickly spread throughout the facility. In one embodiment, the subject in need thereof is suffering from an infectious disease or at risk of suffering from an infectious disease. In particular, said infectious disease is bacterial infection with Gram-positive or Gram-negative bacteria, or a fungal infection, such as Candida albicans infection. Infectious disease and bacterial infections are described in more detail herein below. Salmonella enteritidis infection can lead to substantial mortality and morbidity in young chickens. In particular, the first two weeks after hatching, when their acquired immune system is not yet sufficiently developed broiler chicks are highly susceptible. A possible strategy to improve the health status of broiler chickens is to boost their innate immune memory in order to bridge the gap between fading maternal protection and maturation of adaptive immunity. In one embodiment, the subject, preferably poultry, cattle, pigs, goats or sheep, at risk of suffering from an infectious disease is a subject that is in contact with subjects, preferably poultry, cattle, pigs, goats or sheep, suffering from said infectious disease. A subject is for instance in contact with subjects suffering from an infectious disease if they are kept in the same space, land, stable, house or farm. For instance, once an infectious disease, in particular a bacterial infectious disease or fungal infectious disease, has been established in a farm or stable, treatment of non-infected subjects with CATH2 or derivative thereof in accordance with the present invention is beneficial. In one embodiment, both infected subjects and subjects at risk of suffering from infection, in particular because they are in contact with infected subjects, are treated in accordance with the present invention. Hence, in one embodiment, the CATH2 or derivative thereof is administered to subjects of a population of subjects wherein an infectious disease has been established in one or more subjects of said population. In one embodiment, said subject is preferably poultry or chicken and said population of subjects is preferably a population of poultry or chicken. In another embodiment, said subject is cattle and said population of subjects is a population of cattle. In another embodiment, said subject is a pig and said population of subjects is a population of pigs. Now that it has been found that CATH2 and derivatives induce or promote innate immune memory, it is advantageous to treat both subjects already suffering from the infectious disease and subjects that are at risk of suffering therefrom effectively and concomitantly.

In one embodiment the CATH2 or derivative thereof is comprised in a vaccine. Provided is therefore a vaccine comprising CATH2 or derivative thereof for use in a method for inducing or promoting innate immune memory in a subject in need thereof comprising administering to the subject CATH2 or a derivative thereof. Also provided is therefore a vaccine comprising CATH2 or derivative thereof for use in a method for improving antimicrobial treatment in a subject in need thereof comprising administering to the subject CATH2 or a derivative thereof. As detailed herein below, a vaccine may comprises further constituents, including pharmaceutically acceptable carriers or excipients and one or more adjuvants.

As described herein above, the methods of the invention are particularly suitable to induce or promote innate immune memory for infectious disease and in particular improve antimicrobial treatment. As use herein “infectious disease” is preferably a bacterial infection, viral infection or fungal infection, more preferably a bacterial infection or fungal infection, more preferably a bacterial infection. The bacterial infection can be an infection by both Gram-positive and Gram-negative bacteria, such as E. coli, Salmonella typhimurum, Klebsiella pneumoniae, Haemophilus influenzae, Francisella tularensis, Trueperella pyogenes, Avibacterium paragallinarum, Bacillus anthracis, Bacillus megaterium, Bordetella spp., Brachyspira spp., Brucella spp., Campylobacter spp., Clostridium botulinum, Clostridium perfringens, Clostridium septicum, Trueperella pyogenes, Coxiella burnetii, Enterococcus spp., Haemophilus somnus, Yersinia pestis, Listeria monocytogenes, Mannheimia haemolytica, Mycobacterium tuberculosis, Mycobacterium avium, Mycoplasma gallisepticum, Mycoplasma synoviae, Ornithobacterium rhinotracheale, Pasteurella aeruginosa, Pasteurella multocida, Pneumococcus spp. Pseudomonas aeruginosa, Riemerella anatipestifer, Salmonella spp., Streptococcus uberis, Streptococcus spp., Staphylococcus aureus, Staphylococcus pyogenes, Vibria cholerae, Micrococcus luteus, Moraxella, Neisseria gonnorhoea, Aerobacter spp, Borellia spp. The fungal infection is preferably a Candida albicans infection.

The CATH2 or derivative administered in accordance with the present invention to induce or promote innate immune memory can advantageously be combined with other biologically active agents or excipients. In one embodiment, the CATH2 is administered before, after or simultaneously with a treatment with a pathogenic microorganism or an antigenic part thereof. In one embodiment, the CATH2 or derivative thereof is administered before, after or simultaneously with a treatment with a pathogenic microorganism or an antigenic part thereof which causes a stimulus responsible for training the innate immune cells. A used herein an “antigenic part” of a pathogenic microorganism has the same activity of the microorganism in that it is capable of inducing an immune response against the microorganism. As used herein “before, after or simultaneously with a treatment” means that the administration of CATH2 or derivative and said treatment are preferably maximally 48 hours apart, more preferably maximally 24 hours apart. Any pathogenic microorganism or pathogenic part thereof that is used in the treatment or prevention of infectious disease, such as in a vaccine can be advantageously combined with CATH2 or a derivative thereof in the methods of the present invention. The pathogenic microorganism is for instance an attenuated or inactivated pathogenic microorganism. Such combination advantageously combines induction or promotion of innate immune memory with direct activity and/or specific activity against the pathogenic microorganism. In particular, the pathogenic microorganism is the microorganisms that causes the infectious disease. E.g. the pathogenic microorganism is preferably selected from the group consisting of E. coli, Salmonella typhimurum, Klebsiella pneumoniae, Haemophilus influenzae, Francisella tularensis, Trueperella pyogenes, Avibacterium paragallinarum, Bacillus anthracis, Bacillus megaterium, Bordetella spp., Brachyspira spp., Brucella spp., Campylobacter spp., Clostridium botulinum, Clostridium perfringens, Clostridium septicum, Trueperella pyogenes, Coxiella burnetii, Enterococcus spp., Haemophilus somnus, Yersinia pestis, Listeria monocytogenes, Mannheimia haemolytica, Mycobacterium tuberculosis, Mycobacterium avium, Mycoplasma gallisepticum, Mycoplasma synoviae, Ornithobacterium rhinotracheale, Pasteurella aeruginosa, Pasteurella multocida, Pneumococcus spp. Pseudomonas aeruginosa, Riemerella anatipestifer, Salmonella spp., Streptococcus uberis, Streptococcus spp., Staphylococcus aureus, Staphylococcus pyogenes, Vibria cholerae, Micrococcus luteus, Moraxella, Neisseria gonnorhoea, Aerobacter spp, Borellia spp. In a preferred embodiment, the pathogenic microorganism is selected from the group consisting of a Salmonella species, in particular S. enteritidis, and E. coli.

In the methods of the invention, CATH2 or derivative thereof are further combined with another agent capable of inducing or promoting innate immune memory or an adjuvant specific for innate immunity. Examples of such agents/adjuvants include toll-like receptor (TLR) ligands, ß-glucan, muramyl dipeptide (MDP) or peptide comprising MDP, Bacille Calmette-Guerin (BCG), cytosine-guanine dinucleotide (CpG) containing oligodeoxynucleotide. TLR ligands are known to one of skill in the art and include triacyl and diacyl portions of lipoproteins (TLR2, TLR1, TLR6), flagellin (TLR5), double-stranded RNA (TLR3), single-stranded RNA (TLR7) and bacterial and viral (CpG) DNA (TLR9). MDP is a synthetic peptide conjugate comprising N-acetyl muramic acid and a short amino acid chain of L-alanine D-isoglutamine dipeptide. ß-glucan is a naturally occurring polysaccharide found in the cell wall of yeast, bacteria and fungi. Bacille Calmette-Guarin (BCG) is the vaccine against Mycobacterium tuberculosis (TB). CpG oligodeoxynucleotides are generally present in viral/microbial DNA and are ligand for TLR9 as indicated above.

One aspect of the invention provides a use of CATH2 or a derivative thereof as defined herein as an adjuvant for a pathogen-specific vaccine. As described herein, the recognition of CATH2 and derivatives as innate immune memory inducing peptides allows their use as adjuvants for in particular pathogen-specific vaccines. The pathogen is preferably a pathogenic microorganism, i.e. a pathogenic bacterium, virus, fungus, yeast or parasite. In one embodiment, the pathogen-specific vaccine is an, optionally inactivated or attenuated, pathogen or a pathogen derived peptide or protein, in particular an antigenic pathogen derived peptide or protein. Any pathogen-specific vaccine, which vaccines are known to one of skill in the art, can be advantageously combined with CATH2 or a derivative thereof as defined herein.

As described herein above, the subject that is treated in accordance with the present invention is preferably poultry, such as chicken. Administration of the CATH2 or derivative in accordance with the methods of the invention may be achieved by in ovo administration to poultry embryos or by administration of young poultry after hatch. In the latter case, administration is preferably within one week after hatch, more preferably within 3 days after hatch. As used herein “in ovo administration” refers to administration to eggs of an avian species, preferably eggs in the fourth quarter of incubation. I.e. for chicken eggs, the administration is conducted preferably on about the fifteenth to nineteenth day of incubation, and more preferably on about the eighteenth day of incubation. For turkey eggs, the administration is conducted preferably on about the twenty-first to twenty-sixth day of incubation, and more preferably on about the twenty-fifth day of incubation. Such an administration can be conducted by any method which results in the introduction of one or more of the CATH2 or derivatives into an egg through the shell. A preferred method of administration is by injection. The injection can be performed by using any one of the well-known egg injection devices, such as a conventional hypodermic syringe fitted with a needle of about 18 to 22 gauge, or a high speed automated egg injection system as described in U.S. Pat. Nos. 4,681,063, 4,040,388, 4,469,047, and 4,593,646.

In one embodiment, the subject is administered the CATH2 derivative twice. The two administration are preferably performed with an interval of at least 2 days. In one embodiment, one of the administrations is in ovo administration and one of the administrations is administration after hatch, preferably within one week after hatch, more preferably within 3 days after hatch.

The CATH2 or derivative may be stored in any suitable way, which are known to a person skilled in the art. In one embodiment the CATH2 or derivative is lyophilized, e.g. for storage purposes, e.g. at temperature below 0° C.

The composition comprising the CATH2 or derivative used in accordance with the invention may further comprise an pharmaceutically acceptable carrier, preferably a veterinary acceptable carrier. Such acceptable carrier may include solvents, such as a phosphate buffered saline solution, dispersion media, coatings, adjuvants, stabilizing agents, diluents, preservatives, antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

Adjuvants suitable for use in the present method include but are not limited to: mineral gels, e.g., aluminum hydroxide; surface active substances such as lysolecithin; glycosides, e.g., saponin derivatives such as Quil A or GPI-0100 (U.S. Pat. No. 5,977,081); cationic surfactants such as DDA, pluronic polyols; polyanions; non-ionic block polymers, e.g., Pluronic F-127 (B.A.S.F., USA); peptides; mineral oils, e.g. Montanide ISA-50 (Seppic, Paris, France), carbopol, Amphigen (Hydronics, Omaha, Nebr. USA), Alhydrogel (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol, rmLT, cytokines and combinations thereof. The immunogenic component may also be incorporated into liposomes, or conjugated to polysaccharides and/or other polymers for use in a vaccine formulation. Additional substances that can be included in a product for use in the present methods include, but are not limited to one or more preservatives such as disodium or tetrasodium salt of ethylenediaminetetracetic acid (EDTA), merthiolate, and the like. Immunostimulants which enhance the immune system's response to antigens may also be included in a product. Examples of suitable immunostimulants include cytokines such as IL-12 or IL-2, or stimulatory molecules such as muramyl dipeptide, aminoquinolones, lipopolysaccharide, and the like. In one preferred embodiment, the adjuvant is an adjuvant for innate immune cells, i.e. basophils, dendritic cells, eosinophils, Langerhans cells, mast cells, monocytes and macrophages, neutrophils and/or NK cells, as described herein above: TLR) ligands, ß-glucan, muramyl dipeptide (MDP) or peptide comprising MDP, Bacille Calmette-Guerin (BCG) and CpG containing oligodeoxynucleotide.

In one embodiment, composition comprising the CATH2 or derivative used in accordance with the invention comprises a buffered solution, such as a phosphate buffered saline (PBS) solution, or cholesterol. In one embodiment, composition comprising the CATH2 or derivative used in accordance with the invention comprises a buffered solution, such as a phosphate buffered saline (PBS) solution, and cholesterol. For instance, the cholesterol is first solubilized in ethanol, mixed with PBS and then mixed with the CATH2 or derivative, preferably in dissolved form, resulting in a particulate composition. In one embodiment, prior to administration the dissolved CATH2 or derivative is mixed with a cholesterol solution to form fine particulates and subsequently administered.

A pharmaceutical composition for use in accordance with any method or use of the present invention comprises an effective amount of CATH2 or derivatives as defined herein. As used herein the term “effective amount” refers to an amount of CATH2 or derivative being administered that is sufficient to inducing or promoting innate immune memory in a subject in need thereof as defined herein.

In one embodiment, the composition comprises a therapeutically effective amount of the CATH2 or derivative thereof. The term “therapeutically effective amount,” as used herein, refers to an amount of CATH2 or derivative being administered sufficient to relieve one or more of the symptoms of the disease or condition being treated to some extent, in particular of an infectious disease, preferably a bacterial infection or fungal infection, more preferably a bacterial infection. This can be a reduction or alleviation of symptoms, reduction or alleviation of causes of the disease or condition or any other desired therapeutic effect.

In one embodiment, the composition comprises a prophylactically effective amount of the CATH2 or derivative thereof. As used herein, the term “prophylactically effective amount” refers to an amount of CATH2 or derivative being administered sufficient to preclude or delay the onset of a disease or condition and/or the appearance of clinical symptoms of the disease or condition in a subject that does not yet experience clinical symptoms of the disease, in particular of an infectious disease, preferably a bacterial infection or fungal infection, more preferably a bacterial infection.

The pharmaceutical composition may also comprise CATH2 and one or more derivatives as defined herein, or it may comprise two or more CATH2 derivatives as defined herein, such as a combination of DCATH-2 and D(1-21). The “effective amount”, “therapeutically effective amount” and “prophylactically effective amount” in that case refer to the combined amount of the two or more of CATH2 and/or one or more derivatives thereof.

Effective amounts or dosages of the CATH2 or derivative required for use in a method or use of the invention, can easily be determined by the skilled person, for instance by using animal models. For purposes of the present invention, an effective dose will be from about 0.01 μg/kg to 50 mg/kg, preferably 0.5 μg/kg to about 10 mg/kg of the CATH2 or derivative thereof in the subject to which it is administered. For in ovo applications the same doses may be used, but recalculated with relation to the weight of the embryo.

The pharmaceutical composition may also comprise one or more pharmaceutically acceptable excipients. By “pharmaceutically acceptable” it is meant that the carrier, diluent or excipient must be compatible with the other ingredients of the formulation and not deleterious, e.g. toxic, to the recipient thereof. In general, any pharmaceutically suitable additive which does not interfere with the function of the active compounds can be used. Suitable examples of excipients are a carrier or a diluent. The pharmaceutical compositions may be in the form of a capsule, tablet, lozenge, dragee, pill, droplet, suppository, powder, spray, vaccine, ointment, paste, cream, inhalant, patch, aerosol, and the like. As pharmaceutically acceptable carrier, any solvent, diluent or other liquid vehicle, dispersion or suspension aid, surface active agent, isotonic agent, thickening or emulsifying agent, preservative, encapsulating agent, solid binder or lubricant can be used which is most suited for a particular dosage form and which is compatible with the CATH2 or derivative.

Salts of the CATH2 or derivative may also be used. Salts of peptides can be prepared by known methods, which typically involve the mixing of the peptide with either a pharmaceutically acceptable acid to form an acid addition salt, or with a pharmaceutically acceptable base to form a base addition salt. Whether an acid or a base is pharmaceutically acceptable can be easily decided by a person skilled in the art after taking the specific intended use of the peptide into consideration. Depending on the intended use, pharmaceutically acceptable acids include organic and inorganic acids such as formic acid, acetic acid, propionic acid, lactic acid, glycolic acid, oxalic acid, pyruvic acid, succinic acid, maleic acid, malonic acid, cinnamic acid, sulfuric acid, hydrochloric acid, hydrobromic acid, nitric acid, perchloric acid, phosphoric acid, and thiocyanic acid, which form ammonium salts with free amino groups of peptides and functional equivalents. Pharmaceutically acceptable bases, which form carboxylate salts with free carboxylic groups of peptides and functional equivalents, include ethylamine, methylamine, dimethylamine, triethylamine, isopropylamine, diisopropylamine, and other mono-, di- and trialkylamines, as well as arylamines.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of CATH2 or derivative in either an oil or in aqueous propylene glycol may be employed. The aqueous solutions can be suitably buffered and the liquid diluent can be rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques known to those skilled in the art.

Additionally, it is also possible to administer the CATH2 or derivative topically and this may be done by way of creams, jellies, gels, pastes, patches, ointments and the like, in accordance with standard pharmaceutical practice.

Once formulated, the pharmaceutical compositions can be administered directly to the subject in a method or use in accordance with the invention for inducing or promoting innate immune memory in a subject in need thereof. Also, the pharmaceutical compositions of the invention may be used directly in a method or use of improving antimicrobial treatment in a subject in need thereof. Direct delivery of the compositions will generally be accomplished by forms of administration, including orally, parenterally, subcutaneously, sublingually, intraperitoneally, intravenously or intramuscularly, pulmonary. Such administration may be carried out in single or multiple doses.

It may also be advantageous to administer the CATH2 or derivative in a transmucosal dosage form. This route of administration is non-invasive and thus less cumbersome for the subject that is being treated and at the same time it may lead to an improved bioavailability compared to oral administration, especially if the compound is not stable in the fluids of the digestive system, or if it is too large to be absorbed from the gut effectively. Transmucosal administration is possible, for instance, via nasal, buccal, sublingual, gingival, or vaginal dosage forms. These dosage forms can be prepared by known techniques; they can be formulated to represent nasal drops or sprays, inserts, films, patches, gels, ointments, or tablets. Preferably, the excipients used for a transmucosal dosage form include one or more substances providing for mucoadhesion, thus prolonging the contact time of the dosage form with the site of absorption and thereby potentially increasing the extent of absorption.

In one embodiment, the CATH2 or derivative is administered via the pulmonary route, using a metered dose inhaler, a nebulizer, an aerosol spray, or a dry powder inhaler. Appropriate formulations can be prepared by known methods and techniques. Transdermal, rectal, or ocular administration may also be feasible in some cases.

Pharmaceutical compositions administered in accordance with the invention may contain other active agents, such as conventional antibiotics (like e.g. vancomycin, streptomycin, tetracyclin, penicillin) or other antimicrobial compounds, such as anti-fungals, e.g. itraconazole or myconazole. Also compounds that alleviate other infection symptoms, such as fever (e.g. salicylic acid) or skin rash may be added.

The CATH2 or derivative used in accordance with the invention can be produced synthetically or, where applicable, recombinantly by conventional methods. Suitable methods described in references 10 and 11. Preferably, the CATH2 or derivatives of the invention are prepared conventionally by known chemical synthesis techniques, such as, for instance, are disclosed by Merrifield (J. Am. Chem. Soc. (1963) K:2149-2154). They may be isolated from the reaction mixture by chromatographic methods, such as reverse-phase HPLC.

Alternatively, CATH2 or derivative used in accordance with the invention may be produced by recombinant DNA techniques by cloning and expressing within a host micro-organism or cell a DNA fragment carrying a nucleic acid sequence encoding one of the above-described peptides. Nucleic acid coding sequences can be prepared synthetically, or may be derived from existing nucleic acid sequences (e.g. the sequence coding for wild-type CATH2) by site-directed mutagenesis. These nucleic acid sequences may then be cloned in a suitable expression vector and transformed or transfected into a suitable host cell, such as E. coli, Bacillus, Lactobacillus, Streptomyces, mammalian cells (such as CHO, HEK or COS-1 cells), yeasts (e.g. Saccharomyces, Schizophyllum), insect cells or viral expression systems, such as baculovirus systems, or plant cells. A person skilled in the art will have knowledge of the techniques of constructing the nucleic acid sequences and providing means to enable their expression. Subsequently, the CATH2 or derivative can be isolated from the culture of the host cells. This can be achieved by common protein purification and isolation techniques, which are available in the art. Such techniques may e.g. involve immunoadsorption or chromatography. It is also possible to provide the peptides with a tag (such as a histidine tag) during synthesis, which allows for a rapid binding and purification, after which the tag is enzymatically removed to obtain the active peptide.

Alternatively, the CATH2 or derivative can be produced in cell-free systems, such as the Expressway™ cell-free system of Invitrogen.

Some more comprehensive summaries of methods which can be applied in the preparation of peptides, i.e. including CATH2 or derivative thereof, are described in: W. F. Anderson, Nature 392 Supp., 30 Apr. 1998, p. 25-30; Pharmaceutical Biotechnology, Ed. D. J. A. Crommelin and R. D. Sindelar, Harwood Academic Publishers, 1997, p. 53-70, 167-180, 123-152, 8-20; Protein Synthesis: Methods and Protocols, Ed. R. Martin, Humana Press, 1998, p. 1-442; Solid-Phase Peptide Synthesis, Ed. G. B. Fields, Academic Press, 1997, p. 1-780; Amino Acid and Peptide Synthesis, Oxford University Press, 1997, p. 1-89.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: CATH-2 analogs induce a trained innate immune response in dTHP-1 cells. a Schematic representation of in vitro THP-1 differentiation and training. b Cathelicidin training of dTHP-1 cells differentiated with 100 nM PMA and subsequent re-stimulation with 10 ng/ml S. minnesota LPS after 3 days rest (means±SEM, 3 independent experiments). c In the absence of stimulation, TNFα and IL-6 base levels are not amplified by 24 h cathelicidin training after 3 days rest (means±SEM, 3 independent experiments). LPS-stimulated control cells: 567±166 pg/ml TNFα, 152±18 pg/ml IL-6. d Time-dependency of DCATH-2 training on TNFα production amplification in response to re-stimulation with 1 μg/ml Pam3CSK4. Representative experiment (means±SD, n=6). e DCATH-2 training induced amplification of TNFα and IL-6 production by 100 nM PMA dTH P-1 cells in response to 10 ng/ml E. coli B4:0111 LPS (TLR4), 1 μg/ml Pam3CSK4 (TLR1/2) and 1 μg/ml Pam2CSK4 (TLR2/6). DCATH-2 trained dTHP-1 cells did not result in amplification of CCL5 or CXCL10 production in response to TLR2 and TLR4 agonists. E. coli LPS stimulated control cells: 592±102 pg/ml TNFα, 132±45 pg/ml IL-6, 6.3±2.0 ng/ml CCL5, 16.8±3.3 ng/ml CXCL10, means±SEM, 4 independent experiments. Pam3CSK stimulated control cells: 646±154 pg/ml TNFα, 428±181 pg/ml IL-6, 6.0±3.5 ng/ml CCL5, 3.8±0.6 ng/ml CXCL10, means±SEM, 5 independent experiments. Pam2CSK stimulated control cells: 450±124 pg/ml TNFα, 424±197 pg/ml IL-6, means±SEM, 3 independent experiments. * p<0.05, ** p<0.01, b-d one-way ANOVA with Dunnett's multiple comparison test, e student t-test.

FIG. 2: Trained innate immune response induced by cathelicidins in dTHP-1 cells. Cathelicidin training of dTHP-1 cells differentiated with 8 nM PMA and re-stimulated with 10 ng/ml S. enterica LPS after 3 days rest. Three independent experiments; means±SEM, three independent experiments. LPS-stimulated control cells: 282±63 pg/ml TNFα, 166±30 pg/ml IL-6. * p<0.05, ** p<0.01, one-way ANOVA with Dunnett's test.

FIG. 3: DCATH-2 training enhanced dTHP-1 antimicrobial activity. a Intracellular killing of Salmonella enteritidis 706. b Candidacidal activity against Candida albicans ATCC10231. 3 independent experiments, means±SEM. One-way ANOVA using Dunnett's multiple comparison test. **p<0.01, *p<0.05.

FIG. 4: DCATH-2 training shifts dTHP-1 cell metabolism via mTOR towards aerobic glycolysis. a DCATH-2 training (5 μM) amplified TNFα production by dTHP-1 cells stimulated with 1 μg/ml Pam3CSK4 is inhibited in the presence of mTOR pathway inhibitors. Pam3CSK-induced TNFα production: medium, 304±84 pg/ml; wortmannin, 354±135 pg/ml; rapamycin, 318±45 pg/ml; metformin 387±99 pg/ml; AICAR, 335±89 pg/ml; ascorbate, 421±112 pg/ml (means f SEM, 3 independent experiments). * p<0.05, one-way ANOVA with Dunnett's test. b-h Representative metabolomics experiment (n=3) with 100 nM PMA differentiated dTHP-1 cells primed for 24 h with 5 μM DCATH-2 or medium in the presence or absence of mTOR inhibitor rapamycin (10 nM) followed by 3 times washing and 3 days of rest. b TNFα and IL-6 production after 24 h priming, 3 days of rest and re-stimulation with 50 ng/ml E. coli B4:0111 LPS. c-h Metabolic screen of dTHP-1 cell lysates and culture medium obtained after DCATH-2 or medium priming followed by 3 days of rest. c Lactate production determined in dTHP-1 conditioned culture medium. d Intracellular glucose and lactate concentrations. e-g Heatmaps showing log 2 fold changes in glycolysis (e), pyrimidine metabolism (f) and pentose phosphate pathways (g) relative to unstimulated cells. h L-carnitine and acyl-carnitine levels. Means±SD. * p<0.05, ** p<0.01, *** p<0.001, **** p<0.0001, one-way ANOVA with Tukey's multiple comparisons test.

FIG. 5: DCATH-2 training shifts dTHP-1 cell metabolism towards aerobic glycolysis.

Representative metabolomics experiment (n=3) with 100 nM PMA dTHP-1 cells. Cell lysates and culture medium obtained after DCATH-2 or medium priming (24 h) followed by 3 days of rest. a-e Heatmaps showing log 2 fold changes in metabolites of the TCA cycle (a), urea cycle (b), purine synthesis (c), pyrimidine metabolism (d) and amino acid metabolism (e) relative to unstimulated cells. f Acylcarnitine levels. Means±SD. ** p<0.01, **** p<0.0001, one-way ANOVA with Tukey's multiple comparisons test.

FIG. 6: DCATH-2 training induced metabolic shift is maintained during LPS stimulation.

Representative metabolomics experiment (n=3) with 100 nM PMA differentiated dTHP-1 cells primed for 24 h with 5 μM DCATH-2 or medium in the presence or absence of 10 nM rapamycin followed by 3 times washing, 3 days of rest and 24 h stimulation with E. coli B4:0111 LPS. a Lactate production determined in dTHP-1 conditioned culture medium. b Intracellular glucose and lactate concentrations. c-e Heatmaps showing log 2 fold changes in metabolites of glycolysis (c), pyrimidine biosynthesis (d) and pentose phosphate pathway (e) relative to unstimulated cells. f L-carnitine and acyl-carnitine levels. Means±SD. * p<0.05, ** p<0.01, **** p<0.0001, one-way ANOVA with Tukey's multiple comparisons test.

FIG. 7: DCATH-2 training is not affected by histone methyltransferase inhibitors. dTHP-1 cells were primed with 5 μM DCATH-2 in the absence or presence of broad-spectrum histone methyltransferase (HTM) inhibitor 5′-methylthioadenosine (MTA) and MML1 specific HTM inhibitor, MM-102 before 3 days resting and re-stimulation with 1 μg/ml Pam3CSK4. Pam3CSK-induced TNFα production in the absence of inhibitors: a, 328±45 pg/ml (4 independent experiments, means±SEM); b, 290±37 pg/ml (5 independent experiments, means±SEM). One-way ANOVA using Dunnett's multiple comparisons test. ns: not significant.

FIG. 8: Epigenetic regulation of dTHP-1 cells by DCATH-2 training. dTHP-1 cells were primed with 5 μM DCATH-2 in the absence and presence of histone acetylation transferase inhibitors and after 3 days rest re-stimulated with 1 μg/ml Pam3CSK4. a epigallocathechin gallate (EGCG), b anacardic acid (AA), c curcumin and d garcinol (4 independent experiments, means±SEM). Pam3CSK-induced TNFα production in the absence of inhibitors: a, 290±41 pg/ml; b, 287±38 pg/ml; cd 251±4 pg/ml and e 261±24 pg/ml. * p<0.05, ** p<0.01, *** p<0.001, one-way ANOVA using Dunnett's multiple comparisons test.

FIG. 9: DCATH-2 training of dTHP-1 cells is MAPK p38-dependent dTHP-1 cells were 1 h preincubated with p38 (SB203580), ERK (PD98059), JNK (SP600125) or NFκB (Bay-11-7085) inhibitors before adding 5 μM DCATH-2. Fold increase in TNFα production after 3 days rest and 24 h re-stimulation with 1 μg/ml Pam3CSK4. Representative of three to four experiments, means±SEM. Pam3CSK4-stimulated control cells: 304±84 pg/ml TNFα (p38), 332±68 pg/ml TNFα (ERK), 247±20 pg/ml TNFα (JNK), 261±24 pg/ml (NF-kB). Three independent experiments, means±SEM. One-way ANOVA with Tukey's multiple comparisons test, * p<0.05, ** p<0.01, *** p<0.001.

FIG. 10: Purinergic receptor associated DCATH-2 uptake is necessary for training of dTHP-1 cells. dTHP-1 cells were primed with 5 μM DCATH-2 in the absence and presence of inhibitors of purinergic signaling and endocytosis and re-stimulated with 1 μg/ml Pam3CSK4. a Broad spectrum P2R inhibitor suramin (4 independent experiments, means±SEM) and P2X7R-specific inhibitor KN-62 abrogated and reduced amplification of Pam3CSK4-induced TNFα production (3 independent experiments, means±SEM), respectively. b DCATH-2 training amplified TNFα production was reduced in the presence of lipid raft/caveolae-mediated endocytosis inhibitor nystatin but not by clathrin-mediated endocytosis inhibitor chlorpromazine (3 independent experiments, means±SEM). Pam3CSK4-stimulated control cells: 294±79 pg/ml TNFα (Suramin), 306±93 pg/ml TNFα (KN-62), 260±42 pg/ml TNFα (endocytosis). c Confocal imaging confirmed reduced uptake of TAMRA-labelled D-CATH-2 peptide by dTHP-1 cells in the presence of suramin, KN-62 and nystatin, while uptake was not affected by chlorpromazin. d Violin plots of internalized Tamra-DCATH-2 peptide, expressed as total intensity per cell and puncta intensity per cell in the absence and presence of purinergic receptor and endocytosis inhibitors, 3 independent experiments, median indicated by dotted line. One-way ANOVA using Dunnett's multiple comparison test (a, d) and Kruskal-Wallis with Dunn's multiple comparisons test (d). **P<0.01, ***p<0.001, **** p<0.0001.

FIG. 11: RNA sequencing of DCATH-2 trained dTHP-1 cells. 100 nM PMA differentiated dTHP-1 cells primed for 24 h with 5 μM DCATH-2 or medium followed by 3 times washing, 3 days of rest and 6 h stimulation with E. coli B4:0111 LPS or medium. One-way ANOVA with Tukey's multiple comparisons test, *p<0.05, **** p<0.0001. a Production of TNFα and IL-6 after LPS stimulation. b Heatmap of differentially expressed genes (DEGs) with FDR<0.01 and minimal 2-fold increase relative to controls. c Principle component analysis of all DEGs. d-e Volcano plots of DEGs in DCATH-2 trained dTHP-1 cells in absence of re-stimulation (Dns vs Mns, d) and after LPS re-stimulation (Dlps vs Mlps, e) relative to control cells.

### EXAMPLES

Materials and Methods

Reagents

Salmonella enterica subsp. enterica serovar minnesota R595 LPS (InvivoGen, tlrl-smlps), E. coli serotype O111:B4 LPS (InVivoGen, tlrl-eblps), Pam3CSK4 (InvivoGen, tlrl-pms), Pam2CSK4 (InvivoGen, tlrl-pm2s-1), phorbol 12-myristate 13-acetate (Sigma P8139), mTOR inhibitor rapamycin (Sigma, R 0395), AMPK activators 5-aminoimidazole-4-carboxamide ribonucleotide (AICAR, Sigma A9978), and metformin (InvivoGen, tlrl-meto, Akt inhibitor wortmannin (InvivoGen, tlrl-wtm), HIF-1a inhibitor ascorbate (Sigma, A 4034), MAPK p38 inhibitor SB203580 (Invivogen, tlrl-sb20), ERK inhibitor PD98059 (Sigma, P 215), JNK inhibitor SP600125 (Sigma, S 5567), histone acetyl transferase inhibitors epigallocatechin gallate (EGCG, Sigma E 3768), anacardic acid (Sigma, A 7236), curcumin (Sigma, C 1386), and garcinol (Enzo BML-GR343-0010), NFxB inhibitor Bay-11-7085 (InvivoGen, tirl-b82), histone methyltransferase inhibitor (MTA, Sigma, D 5011), HMTase inhibitor MM-102 (SelleckChem, S7265), P2X/P2Y receptor inhibitor suramin (Sigma, S 2671), P2X7 receptor inhibitor KN-62 (Sigma, I 2142), lipid raft/caveola-mediated endocytosis inhibitor nystatin (Sigma, N 3503), clathrin-mediated endocytosis inhibitor chlorpromazine (Sigma, C 8138).

Peptides

Peptide CATH-2 (Chicken cathelicidin-2; RFGRFLRKIRRFRPKVTITIQGSARF-NH2), its full D-amino acid analog, DCATH-2 (rfgrflrkirrfrpkvtitiqgsarf-NH2), LL-37 (LLGDFFRKSKEKIGKEFKRIVQRIKDFLRNLVPRTES) and N-terminal Tamra-labeled DCATH-2 (TD, TAMRA-rfgrflrkirrfrpkvtitiqgsarf-NH2) were synthesized by FMOC chemistry at CPC scientific (San Jose, CA). Peptides were purified to ≥95% by reversed phase HPLC and checked by mass spectrometry. Peptides were dissolved in LPS-free water (WFI, Gibco), fluorescently labeled peptide was first dissolved in DMSO before further dilution in cell culture media.

Stimulation Experiments

THP-1 cells were cultured in Iscove Modified Dulbecco Media (IMDM, Gibco) containing Glutamax-I, sodium pyruvate, 10% fetal calf serum at 37° C. and 5% CO₂. For differentiation to macrophage-like cells, THP-1 cells were grown in IMDM/FCS medium containing 8 of 100 nM PMA and seeded in 96 wells (5×10⁴ cells/well), 24 wells (3×10⁵ cells/well) or 6 wells plates (1×10⁶ cells/well) during 48 h. Subsequently, dTHP-1 were washed with pre-warmed IMDM/FCS, left overnight to rest and primed during 1 to 24 h with 2-10 μM peptides of fresh IMDM/FCS medium, washed 3 times in medium. After 3 days rest cells were stimulated with various stimuli: S. minnesota LPS (10 ng/ml), E. coli LPS (10 or 50 ng/ml), Pam3CSK4 (1 μg/ml) or Pam2CSK4 (1 μg/ml). After 24 h supernatants were collected and stored at −20° C. For inhibition, dTHP-1 cells were pre-incubated for 1 h before priming and during priming with rapamycin (10 nM), AICAR (50 nM), metformin (0.3 mM), wortmannin (1 μM), ascorbate (50 μM), SB203580 (10 μM), PD98059 (10 μM), SP600125 (10 μM), EGCG (40 μM), anacardic acid (50 μM), curcumin (10 μM), garcinol (10 μM), Bay-11-7085 (10 μM), MTA (1 mM), MM-102 (25 μM), suramin (50 μM), KN-62 (3 μM), nystatin (10 μg/ml), and chlorpromazine (10 μM).

ELISAs

TNFα, IL-6, CXCL10 and CCL5 production were measured using ELISA (R&D systems) following instructions of the manufacturer.

Antibacterial Activity

Antibacterial activity was determined according to Tang et al. [30]. dTHP-1 cells were seeded in 6 well plates and primed with DCATH-2 or medium as described. Log-phase culture of Salmonella enterica subsp. enterica serovar enteritidis 706 (Se706) was added to each well at a MOI of 1. After 2 h incubation at 37° C., cells were washed twice with warm DPBS and further incubated for 1 h incubation at 37° C. with IMDM/FCS medium containing 300 μg/ml colistin. After incubation, cells were washed 3 times in DPBS and the lysed with 1% triton X-100. Well contents were serially diluted in tryptone soy broth (Oxoid), plated on tryptone soy agar and counted after 24 h at 37° C.

Candidacidal Activity

dTHP-1 cells were seeded in 6 well plates and primed with DCATH-2 or medium as described. Candida albicans ATCC10231 was grown in yeast malt broth (Oxoid) at 30° C., diluted in DPBS to 1×10⁴ CFU/ml and added to each well (MOI of 0.03). After 5 h incubation at 30° C., supernatants were transferred and kept. The remaining cells were supplemented with 0.5 ml of sterile water, mixed vigorously and combined with their corresponding well supernatants. Serial dilutions prepared in yeast malt broth were plated onto yeast malt agar. Colonies were counted after 48 h at 30° C.

Confocal Imaging

3×10⁵ dTHP-1 cells were seeded on 8 mm coverslips in 24 wells plates and differentiated for 48 h with 100 nM PMA in 0.5 ml medium. Wells were washed 3 times, and cells were primed for 3 h with Tamra-labelled DCATH-2 (TD) or medium. For inhibition conditions, cells were pre-incubated before priming for 1 h with 3 μM KN-62, 50 μM suramin, 10 μM chlorpromazine, or 10 μg/ml nystatin. After priming, coverslips were washed 3 times with warm medium and fixated for 30 min in 4% paraformaldehyde solution (0.1 M phosphate buffer, pH 7.4). After staining of nuclei with Hoechst (Molecular probes) for 10 min, coverslips were mounted on glass slides using ProLong glass antifade mountant (Thermofisher, P36980). Confocal images were acquired on a Leica SPE-II using the 100×HCX PLAN APO (NA=1.4-0.7) objective. Imaging was performed using a quadruple band beam splitter for the 561 nm laser. Visualization of Hoechst stained dTHP-1 cells was done with a 405 nm (100 mW) Coherent Violet Cube laser. Image analysis was done using composite images of both channels. In brief, regions of interest were set manually using DIC for segmentation. To isolate puncta from background noise, a threshold was set for the Tamra channel and intensity and area of puncta were measured partially automated using a script.

Metabolomics Experiments

dTHP-1 cells were seeded (1×10⁶ cells/well) in 6 wells plates and primed during 24 h with 5 μM DCATH-2 as described using medium as control. Cell supernatants, cell lysates and medium controls were collected before and after 24 h priming in the absence and presence of 10 nM rapamycin, after washing followed by 3 days rest and after subsequent 24 h LPS (E. coli O111:B4) stimulation. For sample collections, cells were washed once in ice-cold PBS, lysed with by adding 1 ml cold methanol/acetonitrile/water (2:2:1) lysis buffer, scraped and transferred in to vials. Cell supernatants and medium control samples (10 μl) were directly mixed with 200 μl lysis buffer. Samples were shaken for 10 min at 4° C. and centrifuged for 15 min at 18,000×g and 4° C. Supernatants were flash frozen in liquid nitrogen and stored at −80° C. for analysis. Liquid chromatography-mass spectrometry analysis was performed using an Exactive mass spectrometer (Thermo Scientific), coupled to a Dionex Ultimate 3000 auto sampler and pump (Thermo Scientific). The mass spectrometry operated in polarity-switching mode with spray voltages of 4.5 and −3.5 kV. Metabolites were separated using a Sequant ZIC-pHILIC column (2.1×150 mm, 5 mm, guard column 2.1×20 mm, 5 mm; Merck) using a linear gradient of acetonitrile and eluent A 20 mM (NH4)2CO3, 0.1% NH40H in ULC/MS grade water (Biosolve BV, Valkenswaard, The Netherlands) and a flow rate of 150 μl/min. Metabolites were identified and peak intensities quantified using LCquan software (Thermo Scientific) on the basis of exact mass within 5 ppm and further validated by concordance with retention times of commercially available standards. Peak intensities were normalized on cell counts of parallel wells before and after LPS stimulation.

RNA Sequencing

dTHP-1 cells (1×10⁶/well) were primed for 24 h with DCATH-2 in 6 well plates containing 1 ml medium, using culture medium as control. After priming, cells were washed 3 times and left to rest for 3 days in culture medium. Cells were stimulated for 6 h with 50 ng/ml E. coli O111:B4 LPS or fresh medium. Supernatants were stored for cytokine analysis. Cells were harvested by rinsing once with ice-cold DPBS and lysing cells in 100 μl RLT buffer (Qiagen) containing 1% 2-mercaptoethanol. After scraping, per condition the contents of two wells were transferred into vials, flash frozen in liquid nitrogen and stored at −80° C. for analysis. Total RNA was extracted in RLT buffer (RNeasy kit, Qiagen) supplemented with 2-mercaptoethanol and purified using a Qiagen QiaSymphony SP system. RNA libraries were prepared using the Truseq stranded total RNA (ribo-zero) library prep kit (Illumina) according the manufacturer's recommendations. RNA-sequencing was done with a NextSeq 500 system 1×75 bp high-output kit.

RNA Sequencing Analysis

Single-end RNASeq reads were processed using the UMCU RNASeq pipeline (v2.3.0) with default settings. Read quality was assessed with FastQC (0.11.4) followed by splice-aware alignment against the human reference genome (GRCh37) with STAR (2.4.2a). RNA expression quantification was performed with htseq-count (0.6.0) in reverse-stranded mode. Differential gene expression analysis was carried out with the DESeq2 package in DEBrowser (https://debrowser.umassmed.edu/). In each comparsion, genes were selected with an absolute fold-change >1.5 and alpha P<0.05. Pathway enrichment analysis was performed in g:Profiler (https://biit.cs.ut.ee/gprofiler/) using separate FDR ranked differentially expressed gene lists for up and down regulated genes with a Benjamini-Hochberg FDR correction for multiple testing and a FDR<0.05 threshold. Enriched pathways of GO Biologic Processes were visualized in Cytoscape (http://www.cytoscape.org/) using EnrichmentMap (http://www.baderlab.org/Software/EnrichmentMap) and a FDR cutoff of 0.01. Similarity statistic threshold was set at Jaccard>0.25 and filtered for gene set sizes between 5 and 500 genes [61].

Statistical Analysis

Differences between groups were calculated with one-way ANOVA using Dunnett's or Tukey's multiple comparison test. Alternatively, Kruskal-Wallis test was used. Levels of significance were defined as p<0.05 (*), p<0.01 (**), p<0.001 (***) or p<0.0001 (****).

Results

Innate Immune Training of dTHP-1 Cells by CATH-2 Analogs.

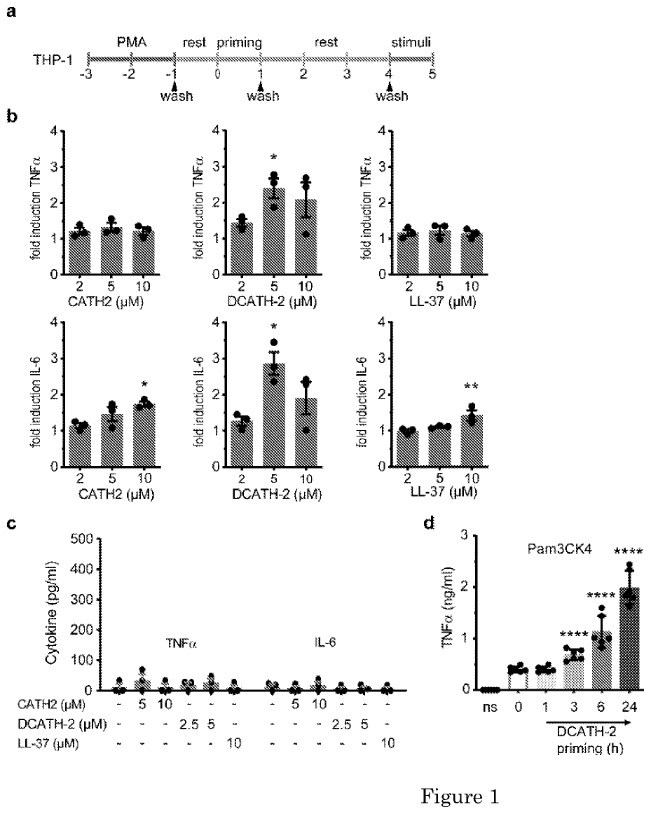

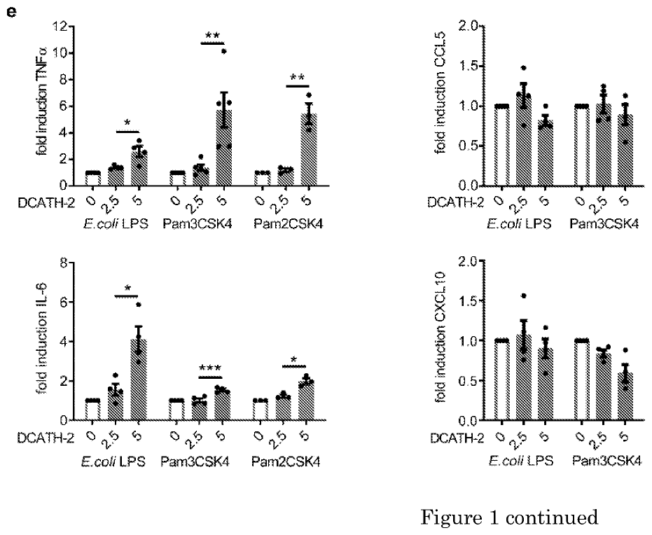

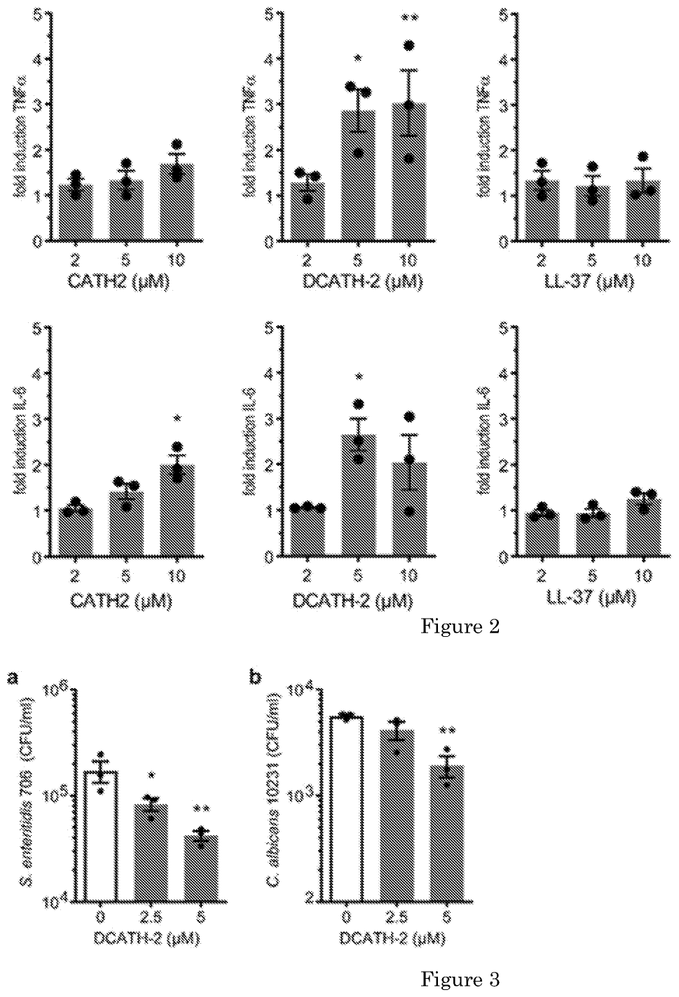

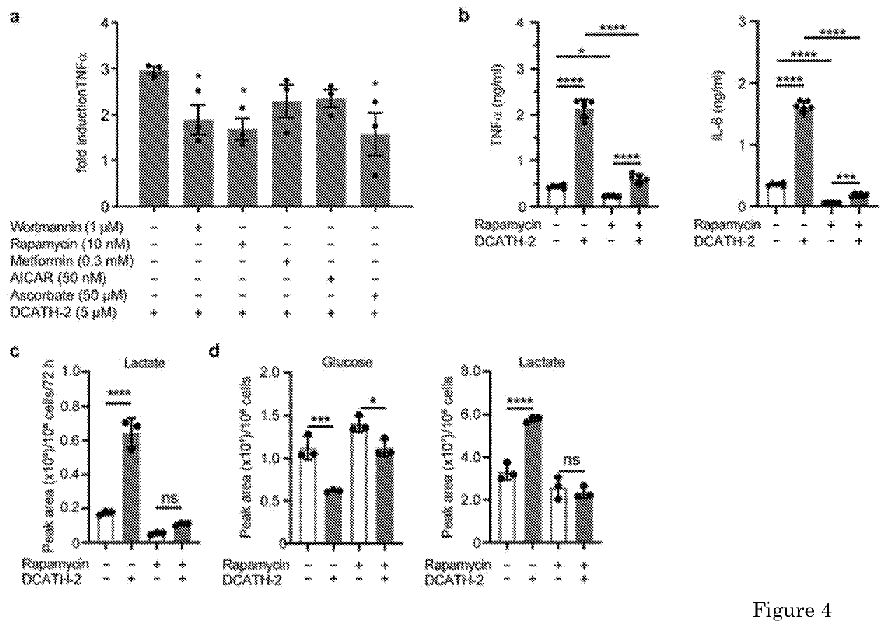

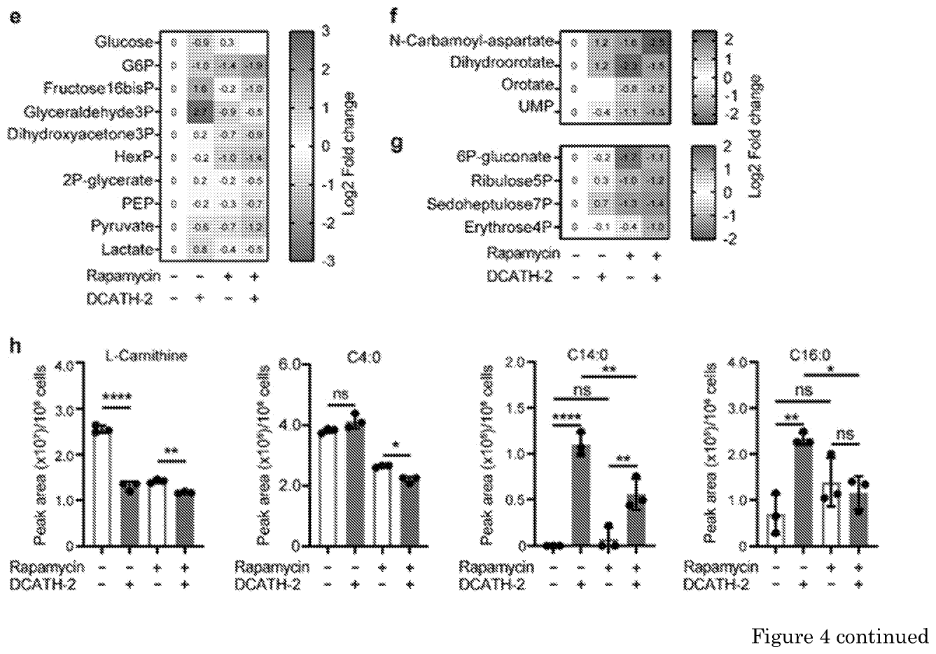

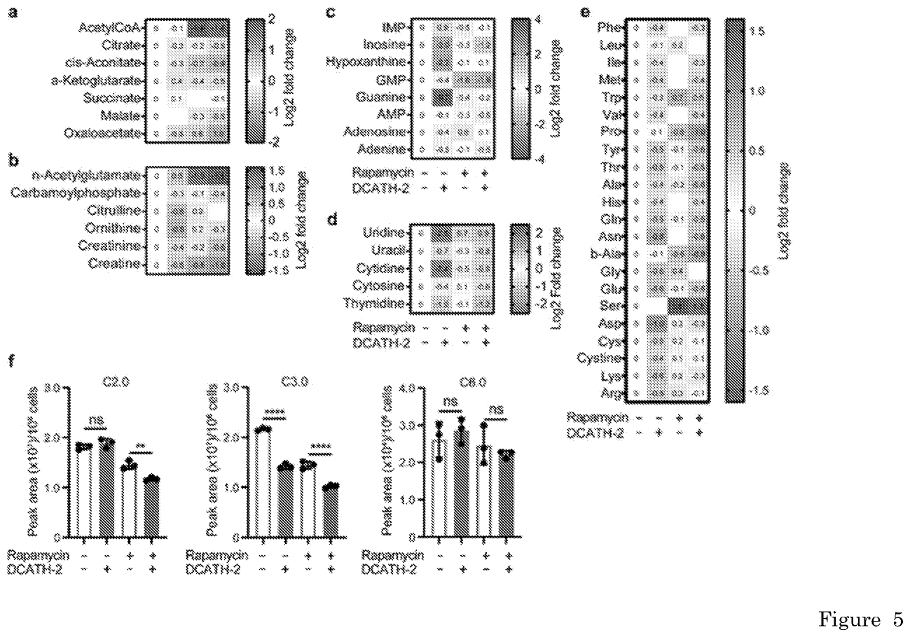

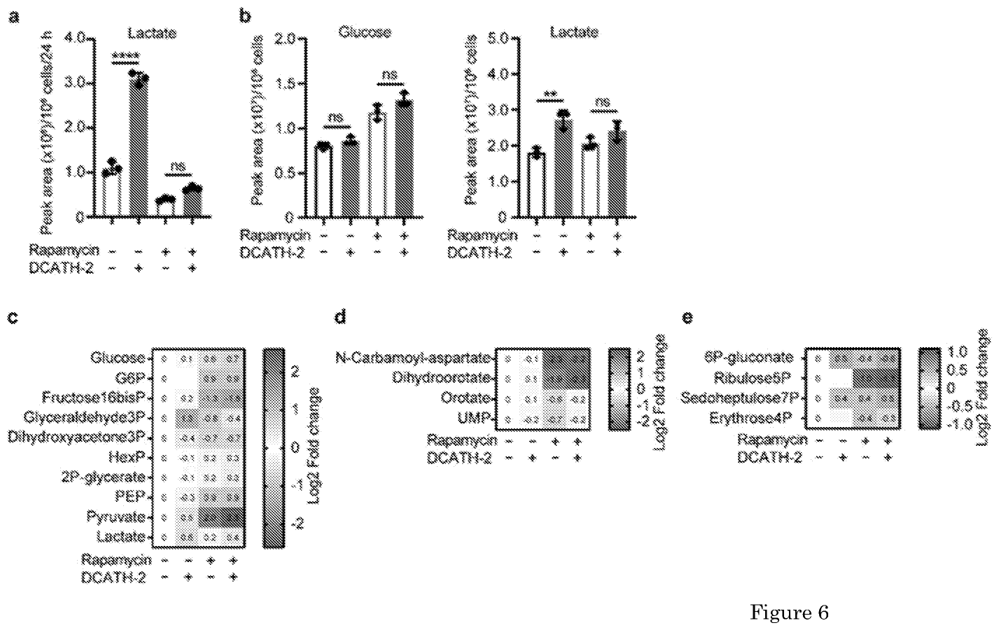

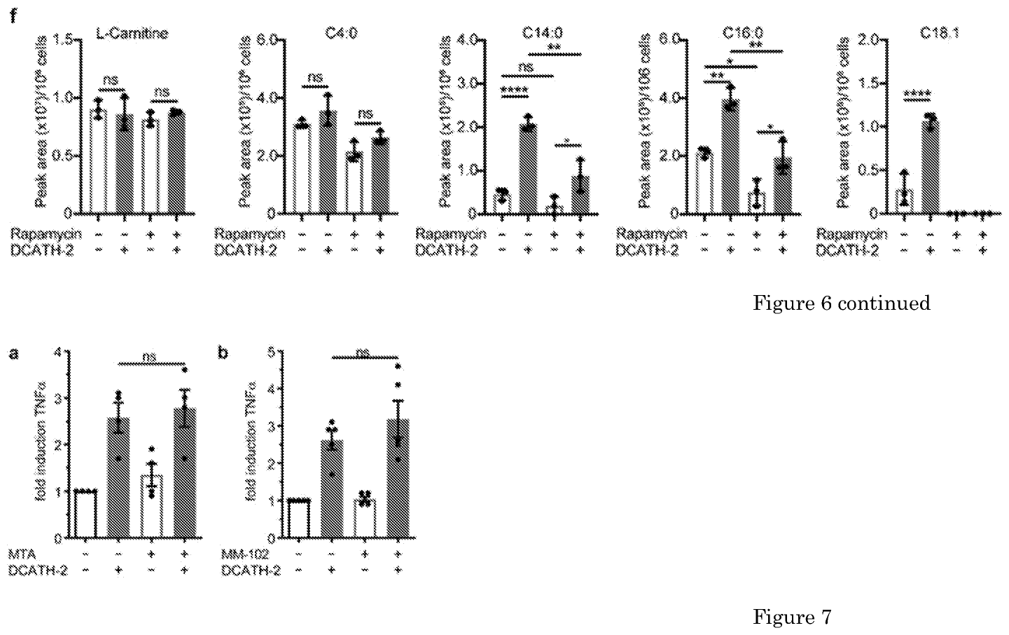

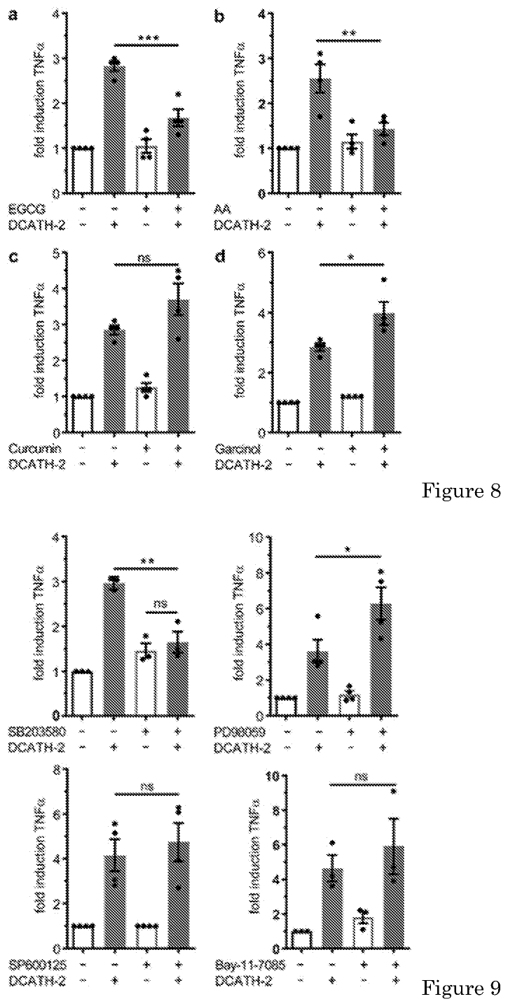

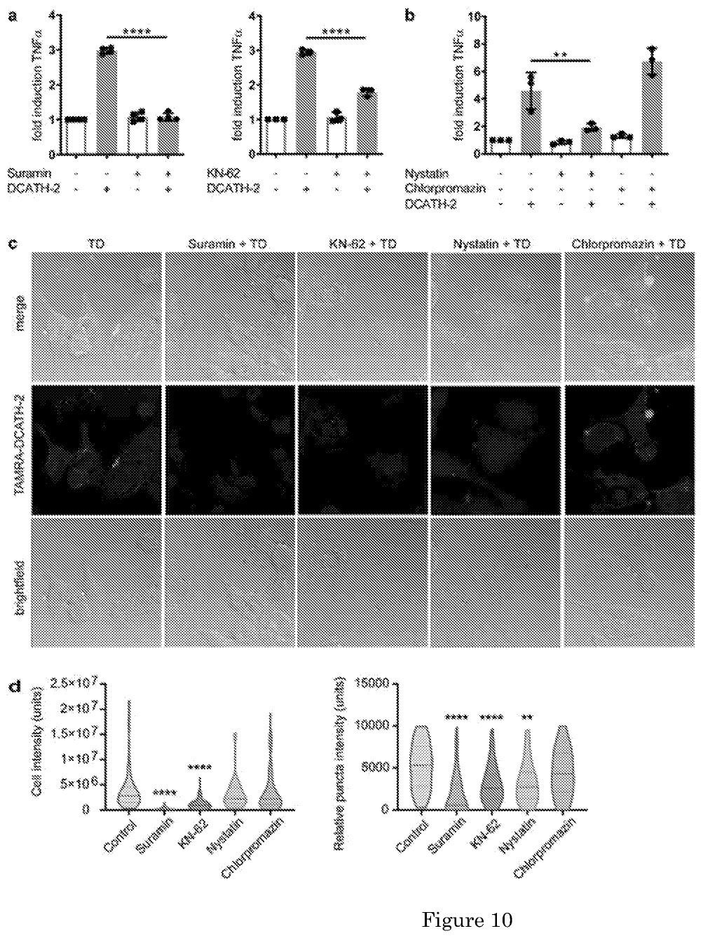

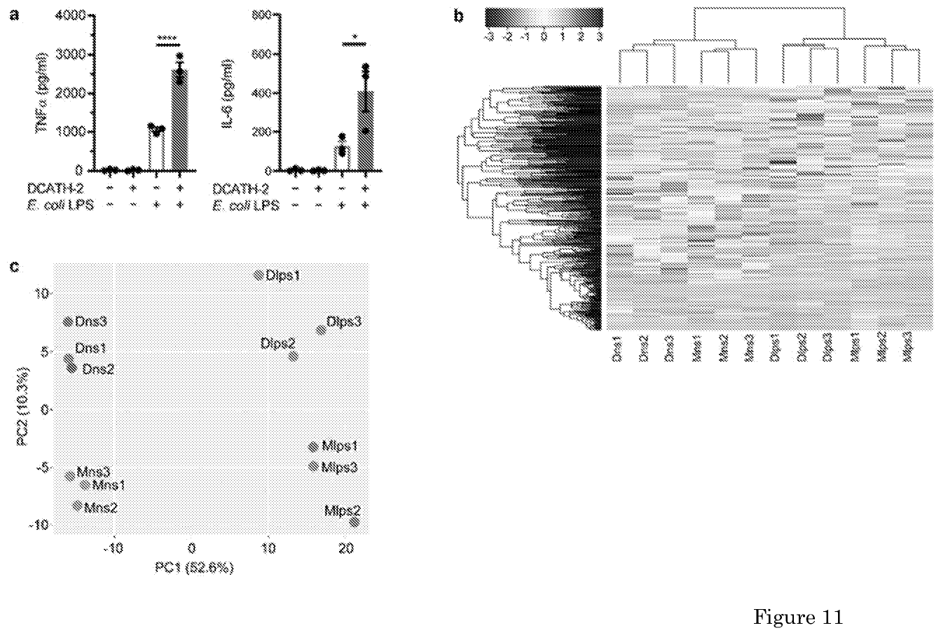

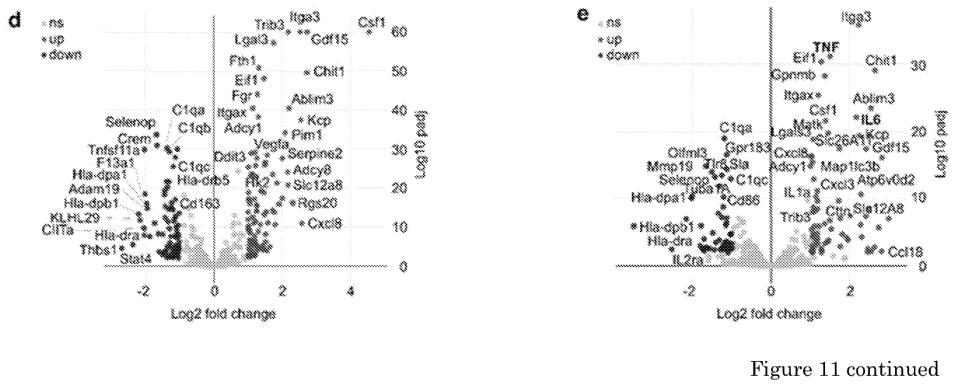

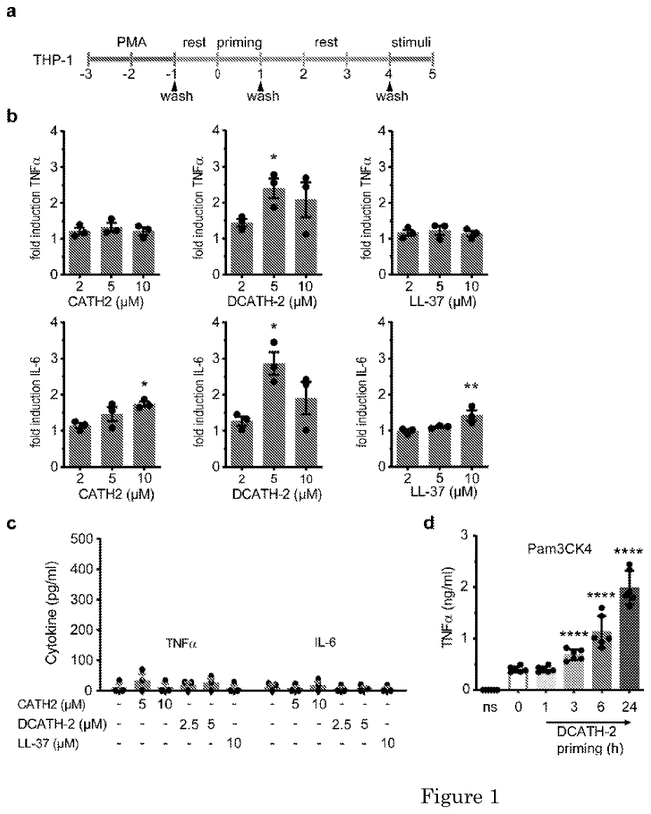

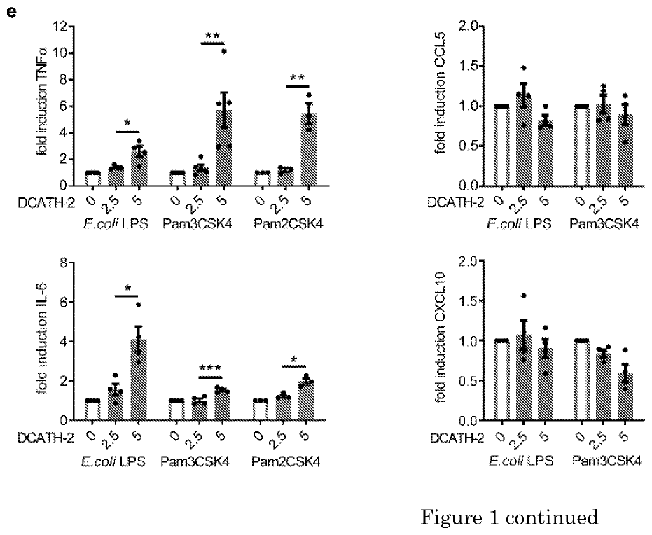

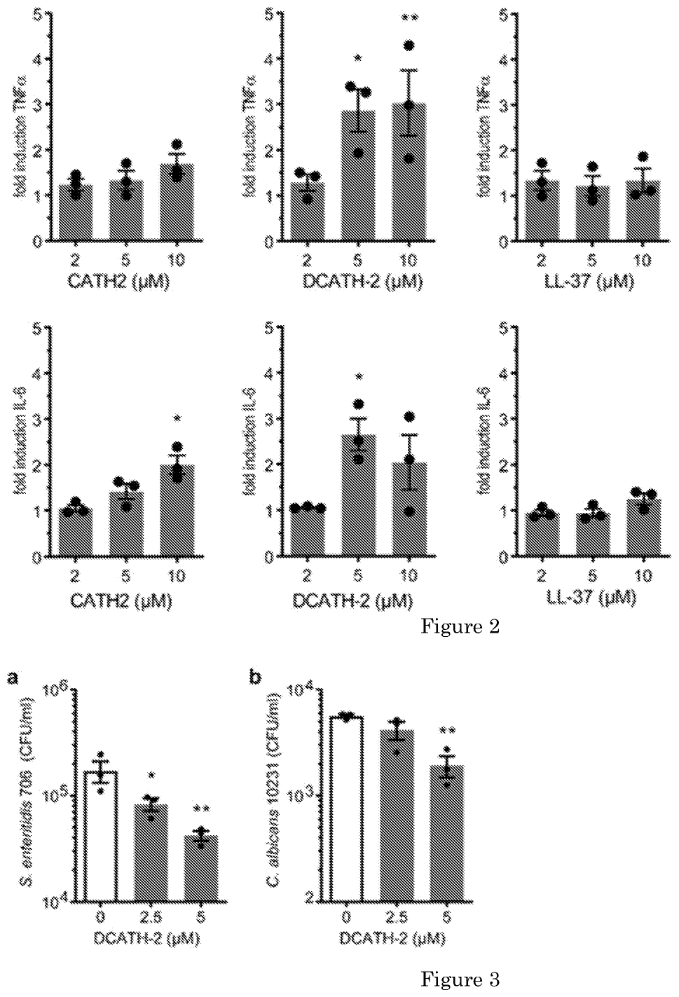

Extrapolating from the trained immunity experiments with human monocytes [2, 3, 20], we examined if mature cathelicidin peptides could induce trained immunity in TH P-1 cells, a human monocyte cell line. TH P-1 cells were differentiated with 100 nM or 8 nM phorbol 12-myristate 13-acetate (PMA) during 48 h washed 3 times and, after overnight rest, primed with 2-10 μM of chicken cathelicidin 2 (CATH-2), its full D-analog DCATH-2 or human cathelicidin LL-37 for 24 h. Subsequently, dTHP-1 cells were washed 3 times and re-stimulated after a 3-day rest period with TLR agonists (24 h) (FIG. 1a). Priming with 10 μM CATH-2 resulted in a 1.8-fold amplification of IL-6 production when re-stimulated with 10 ng/ml Salmonella minnesota LPS for 100 nM PMA dTHP-1 cells (FIG. 1b). Priming with 5 μM of its full-D-amino acid analog DCATH-2 amplified 2- to 3-fold the TNFα and IL-6 production in response to LPS stimulation. A minor response was found for priming with 10 μM LL-37, which amplified IL-6 production 1.4-fold in 100 nM differentiated cells. Differentiation of THP-1 with a lower concentration of PMA has been suggested to result in lower basal levels of pro-inflammatory cytokine production [21] and might affect training efficiency. However, similar responses were found for CATH-2 and DCATH-2 training of 8 nM PMA differentiated THP-1 cells (FIG. 2).

DCATH-2 Trained Immunity Amplifies Both TLR2 and TLR4 Activation

Basal production levels of TNFα and IL-6 were not altered by 24 h priming with 2.5-5 μM DCATH-2 (FIG. 1c). DCATH-2 training of dTHP-1 cells was time-dependent, reaching a highest level of training after 24 h (FIG. 1d) similar to training of primary monocytes with ß-glucan [2]. Next, we examined if DCATH-2 training affected the dTHP-1 response to different TLR2 and TLR4 ligands. dTHP-1 training with 5 μM DCATH-2 followed by re-stimulation with E. coli O111:B4 LPS, Pam3CSK4 or Pam2CSK4 resulted in amplified TNFα and IL-6 production (FIG. 1e). The production of CCL5 and CXCL10 was not altered by DCATH-2 training (FIG. 1e). Thus, stimulation by rough (FIG. 1b, FIG. 2) and smooth LPS (TLR4), triacyl- (TLR1/2) and diacyl lipopeptides (TLR2/6) all lead to an amplified pro-inflammatory response by DCATH-2 trained dTHP-1 cells.

DCATH-2 Trained dTHP-1 Cells have Increased Antimicrobial Killing Capacity

Intracellular pathogens successfully attempt to evade or exploit the host innate immune system [22-24]. We found that DCATH-2 training (24 h) of dTHP-1 cells enhanced their antimicrobial activity against Salmonella enteritidis infection and Candida albicans infection (FIG. 3). Compared to non-primed dTHP-1 cells, DCATH-2 primed dTHP-1 cells inhibited outgrowth of Salmonella enteritidis after 24 h by 78±11% and Candida albicans after 48 h by 68±10%.

DCATH-2 Training Utilizes the Akt-mTOR-HIF1α Signaling Pathway

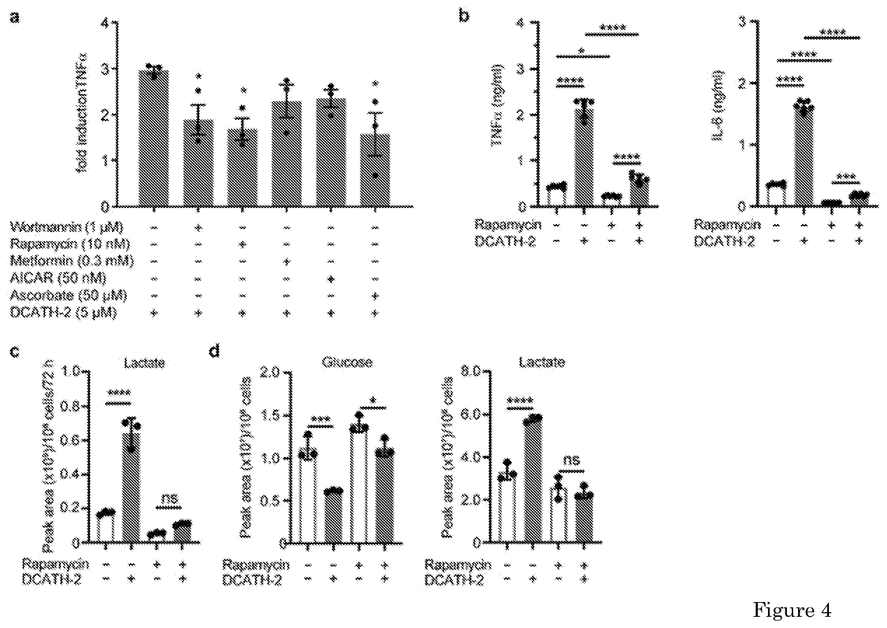

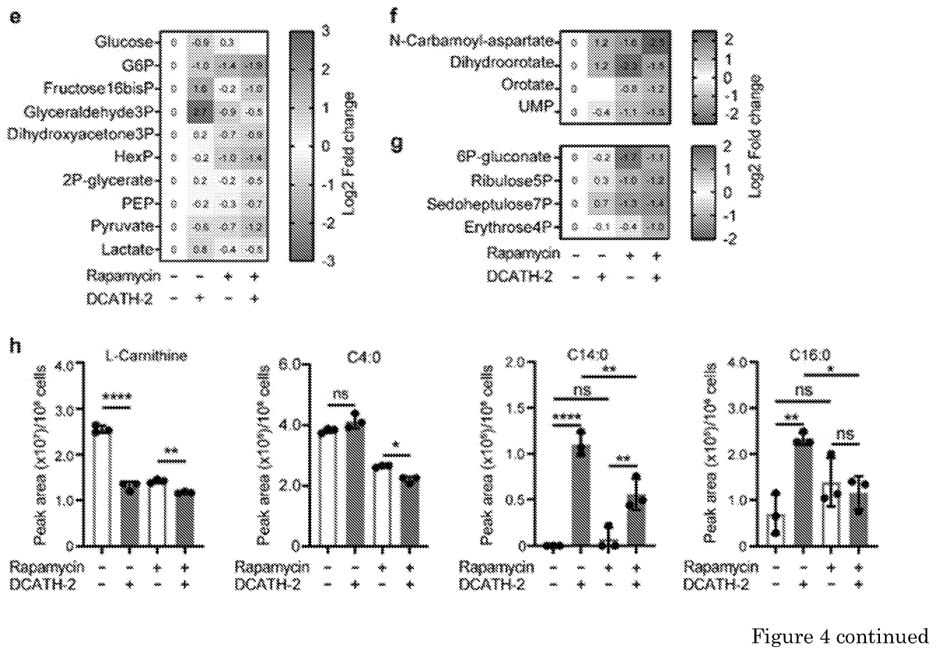

Training of human monocytes is known to induce a shift in the cell metabolism from oxidative phosphorylation towards aerobic glycolysis mediated via the Akt-mTOR-HIF1α pathway [6]. To examine the involvement of the Akt-mTOR-HIF1α pathway in DCATH-2 training, dTHP-1 cells were pre-incubated with mTOR pathway specific inhibitors prior to priming with DCATH-2 peptide. Direct inhibition of Akt (wortmannin), mTOR (rapamycin) and HIF1α (ascorbate) strongly interfered with DCATH-2 training of dTHP-1 cells (FIG. 4a) indicating that DCATH-2 utilizes similar pathways as ß-glucan to shift cell metabolism towards aerobic glycolysis. Indirect mTOR inhibition by AMPK activation with metformin and AICAR during DCATH-2 training did not result in a significant reduction of Pam3CSK-induced TNFα production.

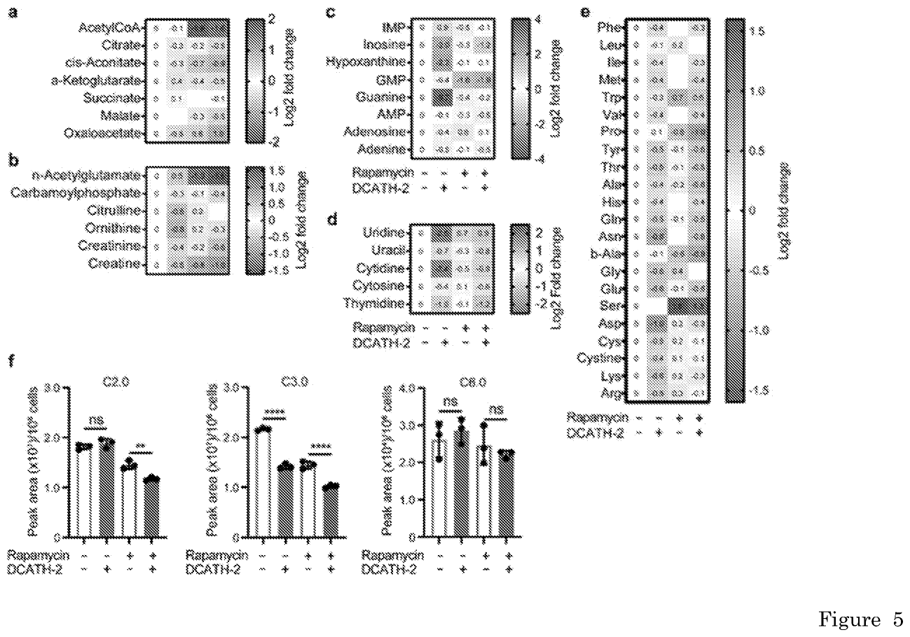

mTOR/Akt signaling controls the metabolism and activation of macrophages with mTORC1 upregulating rate-limiting enzymes in glycolysis, fatty acid synthesis and the pentose phosphate pathway (PPP) via SREBP1 and nucleotide synthesis via increased CAD activity, a rate-limiting enzyme in pyrimidine synthesis [25]. To examine the impact of DCATH-2 training on dTHP-1 cells, metabolome analysis was performed using culture medium and cell lysates of DCATH-2 trained and untrained dTHP-1 cells after 3 days rest. DCATH-2 amplified TNFα and IL-6 production upon E. coli LPS stimulation was strongly reduced by the presence of rapamycin during priming (FIG. 4b). As expected for increased aerobic glycolysis, DCATH-2 training augmented lactate secretion into the medium (FIG. 4c). In line with these results, intracellular lactate levels were higher and intracellular glucose concentrations were lower in DCATH-2 trained cells (FIG. 4d). Analysis of metabolic intermediates indicated that DCATH-2 training increased glycolysis (FIG. 4e), to a smaller extent the pentose phosphate pathway (FIG. 40 and increased de novo pyrimidine synthesis (FIG. 4g), supporting involvement of enhanced mTOR activation. Rapamycin presence during DCATH-2 priming abolished augmentation of lactate production, glycolysis, pentose phosphate pathway and de novo pyrimidine synthesis (FIG. 4b-g), confirming that DCATH-2 trained immunity is accompanied by an mTOR regulated shift towards aerobic glycolysis. The TCA cycle and urea cycle were not significantly affected by DCATH-2 training (FIG. 5a-c). DCATH-2 training resulted in an overall enhanced usage of amino acids suggesting increased protein synthesis (FIG. 5d). DCATH-2 training dramatically increased levels of intracellular medium-chain and long-chain acylcarnitines (FIG. 5f) indicating decreased transport of fatty acids into the mitochondria.

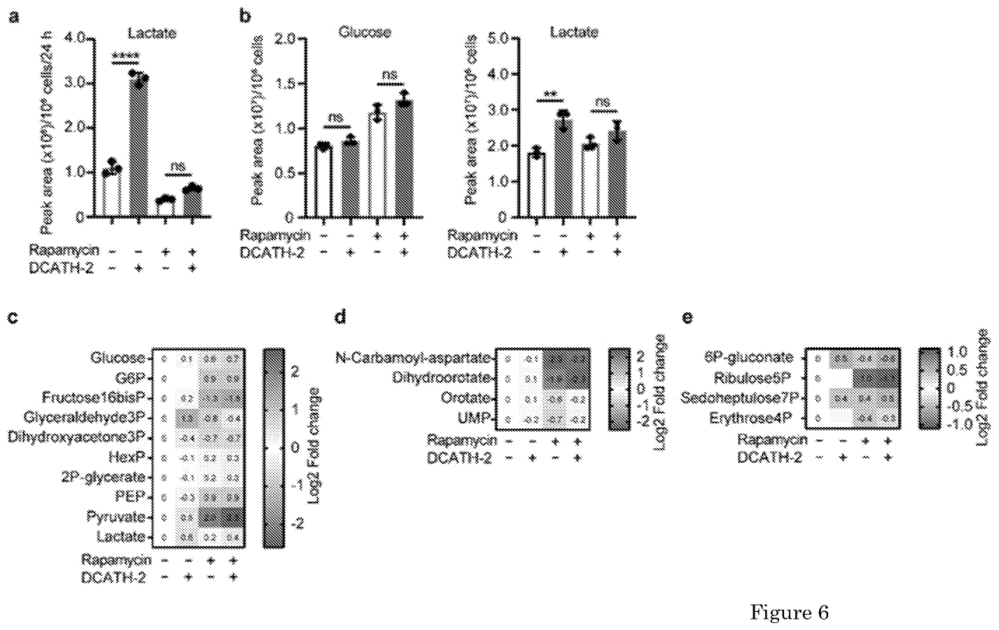

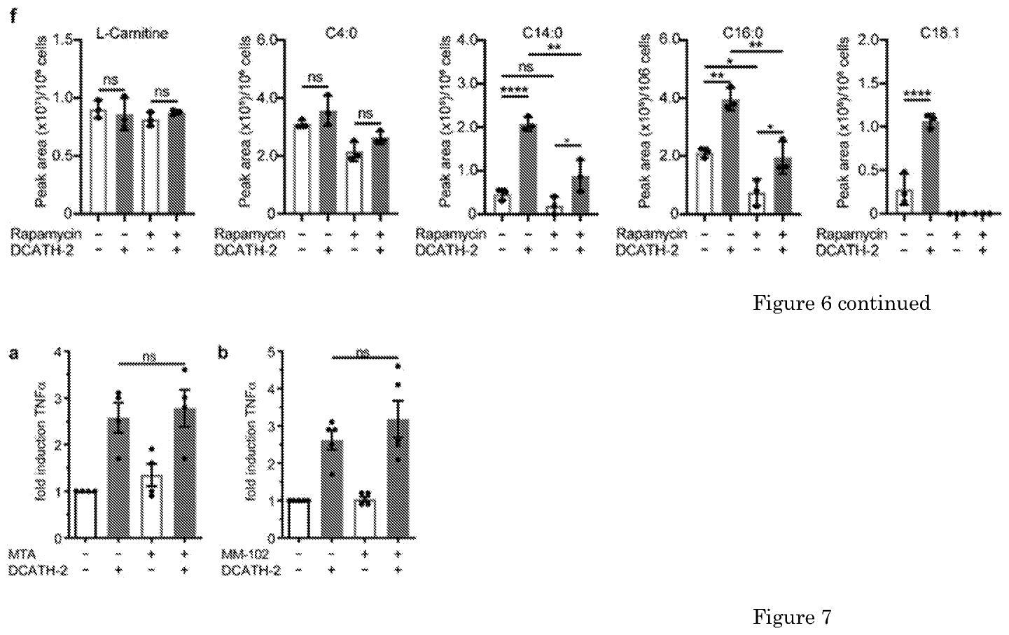

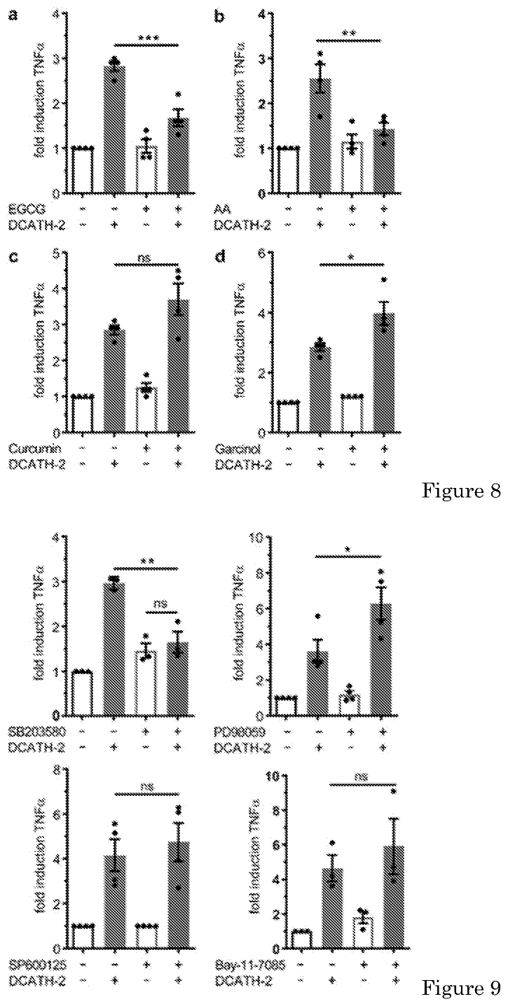

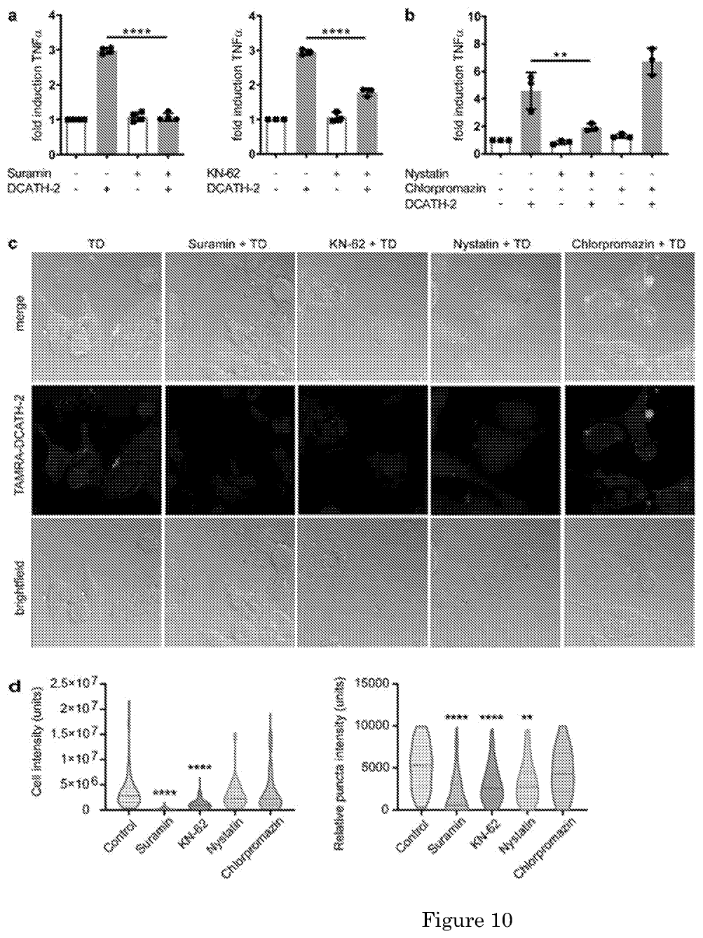

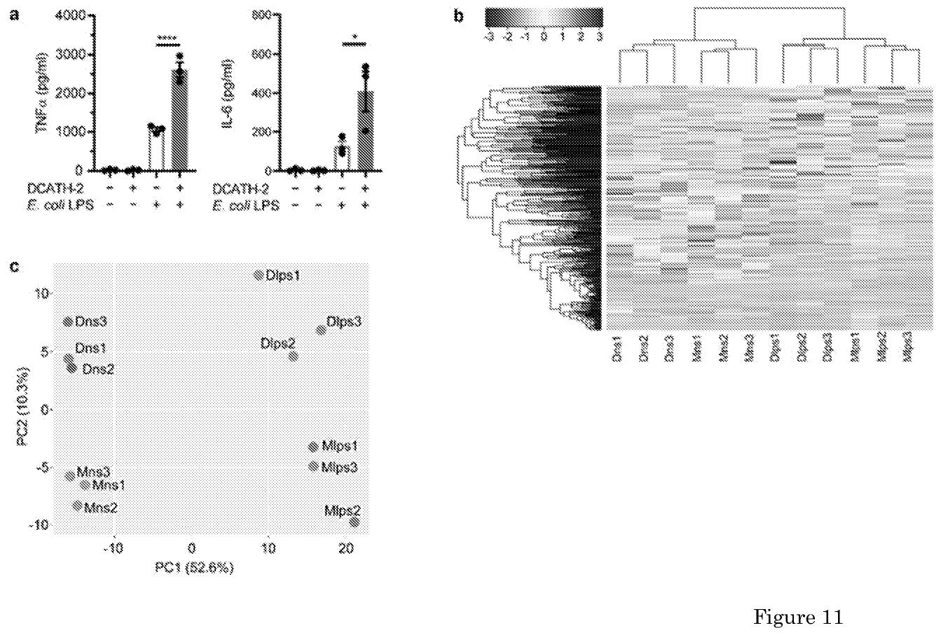

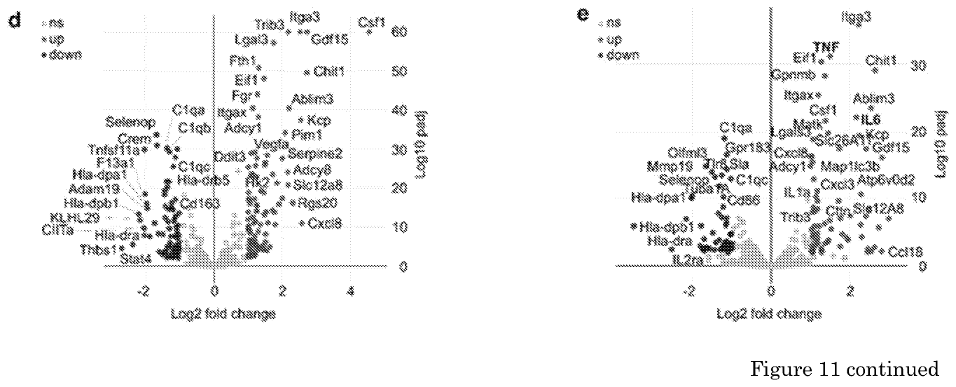

After 24 h LPS stimulation, DCATH2 trained cells still exhibited enhanced glucose uptake and lactate production, increased glycolysis and PPP pathway metabolites albeit at a lower extent (FIG. 6a-e). Intracellular levels of medium-chain and long-chain acylcarnitines remained elevated in DCATH-2 trained cells (FIG. 60. Rapamycin reduced DCATH-2 training-induced effects on cell metabolism during LPS stimulation (FIG. 6). LPS stimulation of DCATH-2 trained cells did not affect the TCA cycle, urea cycle or amino acid metabolism (FIG. 5a-e). Thus, the DCATH-2 training induced metabolic shift is maintained during LPS stimulation.

DCATH-2 Training of dTHP-1 Cells is Epigenetically Regulated

DCATH-2 induced training augmented TLR2/4 ligand induced TNFα and IL-6 production without changing basal TNFα and IL-6 production, suggesting that DCATH-2 priming induced epigenetic reprogramming of dTHP-1 cells. Enriched H3K4me3 levels at promoters of immune-related genes including TNFα and IL-6 were found for human monocytes trained with BCG [3], ß-glucan [2] or oxidized LDL [20] which positively related to enhanced transcription upon secondary stimulation. Increased H3K27ac levels in cytokine encoding genes were found in monocytes isolated after BCG vaccination and in ß-glucan trained monocytes [6, 26]. To determine the role of histone acetylation in DCATH-2 training, dTHP-1 cells were primed in the presence of histone acetyltransferase (HAT) inhibitors curcumin (10 μM), garcinol (10 μM), epigallocathechin gallate (EGCG, 40 μM) and anacardic acid (50 μM). It was found that DCATH-2 training of dTHP-1 cells was abrogated by histone acetyl transferase (HAT) inhibitors EGCG and anacardic acid, but not by garcinol or curcumin (FIG. 8a-d). Neither methyltransferase inhibitor MTA (5′-methylthioadenosine), nor MLL1 inhibitor MM-102 prevented amplification of Pam3CSK4-induced TNFα production in DCATH-2 primed cells (FIG. 7).

Trained Immunity by DCATH-2 Requires MAPK p38 Signaling

Both p38 and ERK are important signaling pathways for regulation of pro-inflammatory cytokines such as TNFα [27, 28] and IL-6 [29] and MAPK p38-mediated signaling is involved in trained immunity with ß-glucans [1, 2]. To define the involvement of MAPKs in the DCATH-2 training, dTHP-1 cells were with the specific inhibitors of p38 (SB203580), ERK (PD58059) and JNK (SP600125). Inhibition of MAPK p38 completely blocked DCATH-2 trained enhanced Pam3CSK-induced TNFα production, whereas ERK and JNK inhibition had no effect (FIG. 9). Pre-incubation with 10 μM NF-κB inhibitor Bay-11-7085 did not impair DCATH-2 training of dTHP-1 cells (FIG. 9).

D-CATH-2 Training of dTHP-1 Cells is Mediated by Purinergic Receptors

Immunomodulatory functions of cathelicidins have been associated with different G-protein coupled receptors such as epidermal growth factor receptor (EGFR), formyl peptide receptor (FPR) and purinergic receptor P2X7R [30]. Although P2X7R activation is primarily triggered by high levels of ATP, other endogenous ligands including the cathelicidin LL-37 are able to interact and activate P2X7R [31]. To examine the involvement of P2X7R in cathelicidin training, dTHP-1 cells were pre- and co-incubated with the P2 family inhibitor suramin or with P2X7 inhibitor KN-62. Suramin completely impaired DCATH-2 training-induced enhanced production of TNFα, whereas this was partially inhibited by P2X7R inhibitor KN-62 (FIG. 10a). Internalization of LL-37 by monocytes is known to be P2X7-dependent, and occurs via clathrin- and caveolae/lipid raft-mediated endocytosis [30]. A function of the P2X7 receptor in training may therefore be to facilitate uptake of DCATH-2 peptide by monocytes/macrophages. To test this hypothesis, dTHP-1 cells were pretreated with endocytosis inhibitors nystatin (caveolae/lipid raft-mediated) and chlorpromazin (clathrin-mediated). Pam3CSK4-induced TNFα production was strongly impaired for nystatin treated cells but not affected by chlorpromazin (FIG. 10b) suggesting that DCATH-2 training of dTHP-1 cells requires uptake via lipid raft-mediated endocytosis. Confocal imaging analysis of TAMRA-labelled DCATH-2 uptake by dTHP-1 cells in the presence of suramin, KN-62 or nystatin showed a reduction of overall signal intensity in the presence of P2/P2X7R inhibitors and a reduced relative intensity in puncta for P2/P2X7R inhibitors as well as for caveola/lipid raft-mediated endocytosis (FIG. 10c,d). TAMRA-DCATH-2 uptake was not reduced by inhibition of clathrin-mediated endocytosis. These findings confirmed that DCATH-2 training correlates with caveolae/lipid raft-mediated uptake by dTHP-1.

DCATH-2 Training Skewed Transcription is Sustained During Re-Stimulation

To elucidate which biological processes were persistently altered by DCATH-2 training of dTHP-1 cells, the transcriptome (RNAseq) of DCATH-2 primed cells was compared with that of control cells after 3 days rest and after 6 h LPS stimulation. LPS stimulation resulted in a 2.5-fold and 3.3-fold amplification of TNFα and IL-6 production (FIG. 11a). Hierarchical clustering of RNAseq data showed subsets of genes that remained altered by DCATH-2 priming after 3 days rest (FIG. 11b). Principle component analysis indicated that most variation between differentially expressed genes (DEGs) could be explained by LPS stimulation (52.6%) and DCATH-2 training (10.3%) (FIG. 11c). 156 up- and 126 downregulated DEGs were identified for trained and rested dTHP-1 cells compared to unstimulated control cells. Following LPS stimulation, 71 upregulated and 54 downregulated DEGs were identified in DCATH-2 trained LPS-stimulated cells relative to LPS-stimulated control cells. Comparison of genes using volcano plots revealed a subset of genes uniquely upregulated/induced by LPS stimulation in trained cells (FIG. 11d,e, table 1) and were related to events occurring extracellular and at the plasma membrane and associated with enhanced cellular responses to cytokine stimulus, cytokine-mediated signaling and cytokine/chemokine receptor binding.

TABLE 1
Genes uniquely upregulated after DCATH-2
training during LPS stimulation.

| | | | Log 2 |
|---|---|---|---|
| | | Adjusted | fold |
| Gene | Description | p-value | change |
| IL6 | Interleukin-6 | 7.47E−23 | 2.17 |
| MFSD2A | Major Facilitator Superfamily | 4.83E−17 | 1.04 |
| | Domain Containing 2A | | |
| CXCL3 | C-X-C Motif Chemokine Ligand 3 | 6.93E−12 | 1.18 |
| IL1A | Interleukin-1A | 4.58E−11 | 1.15 |
| TNFSF15 | TNF Superfamily Member 15 | 2.24E−10 | 1.19 |
| TRPM8 | Transient Receptor Potential Cation | 5.84E−07 | 1.36 |
| | Channel Subfamily M Member 8 | | |
| FOXF1 | Forkhead Box F1 | 1.31E−06 | 1.21 |
| LIF | LIF Interleukin 6 Family Cytokine | 2.03E−06 | 2.73 |
| MYLK | Myosin Light Chain Kinase | 1.35E−05 | 1.17 |
| CSF2 | Colony Stimulating Factor 2 | 1.88E−05 | 1.81 |
| CD22 | CD22 Molecule | 7.46E−05 | 1.18 |
| TIE1 | Tyrosine Kinase With Immunoglobulin | 1.61E−04 | 1.46 |
| | Like And EGF Like Domains 1 | | |
| AHNAK2 | AHNAK Nucleoprotein 2 | 1.77E−04 | 1.12 |
| CCRL2 | C-C Motif Chemokine Receptor Like 2 | 1.01E−03 | 1.04 |
| ZNF358 | Zinc Finger Protein 358 | 4.52E−03 | 1.04 |
| HSPBAP1 | HSPB1 Associated Protein 1 | 5.56E−03 | 1.06 |
| SEPT3 | Septin 3 | 6.92E−03 | 1.06 |
| KLF2 | Kruppel Like Factor 2 | 6.96E−03 | 2.54 |
| CCL18 | C-C Motif Chemokine Ligand 18 | 7.05E−03 | 2.81 |
| FAM213B | Peroxiredoxin Like 2B | 7.63E−03 | 1.02 |
| TRIM47 | Tripartite Motif Containing 47 | 8.53E−03 | 2.45 |
| PTGS2 | Prostaglandin-Endoperoxide Synthase 2 | 9.60E−03 | 1.29 |

Pathway Analysis of Biological Processes Influenced by DCATH-2 Training

To map the biological processes altered by DCATH-2 priming after 3 days rest, pathway enrichment analysis was performed with G:profiler [32] and visualized using Cytoscape [33] and Enrichment Map [34]. G:profiler analysis of differentially expressed genes revealed 590 upregulated and 612 downregulated GO biological processes (FDR<0.05) in DCATH-2 trained unstimulated dTHP-1 cells. Enriched pathways (FDR<0.01) were visualized separately using EnrichmentMaps in Cytoscape and clustered within themes using autoannotation. Major upregulated themes were cellular response to stimulus, transcription and translation. Minor upregulated themes were signal transduction, autophagy and metabolism (table 2). Upregulated pathways were associated with oxidative stress, endoplasmic reticulum (ER) stress and unfolded protein response (UPR) linked to enhanced transcription and translation. Heatmaps of leading edge gene expression confirmed upregulation of nonsense-mediated mRNA decay, PERK-mediated unfolded protein response (UPR), positive regulation of RNA polymerase II to stress and ribosome biogenesis. Most prominent downregulated processes were clustered around antigen processing and presentation (table 2).

Pathway Analysis of Biological Processes of LPS-Stimulated Trained dTHP-1 Cells

Next, we investigated how DCATH-2 training affected biological processes under inflammatory conditions. G:profiler analysis of DEGs (FDR<0.05) resulted in 322 upregulated and 416 downregulated GO biological pathways in DCATH-2 trained LPS-stimulated cells relative to untrained control cells. Enriched pathways (FDR<0.01) revealed upregulated biological processes associated with response to stimulus, signal transduction and immune response, differentiation and metabolism (table 3). Downregulated pathways were related to antigen presentation and metabolism (table 3).

TABLE 2
Pathway enrichment analysis of DCATH-2 primed unstimulated dTHP-1 cells.

| | | | | Gene | |
|---|---|---|---|---|---|
| | | Gene | | set | FDR |
| Cluster | Biological theme | ontology | GO biological process | size | Q-value |
| Upregulated | | | | | |
| cotranslational targeting ER | Translation | GO:0000184 | nuclear-transcribed mRNA catabolic process, | 119 | 1.40E−26 |
| | | | nonsense-mediated decay | | |
| polymerase ii promotor | Transcription | GO:0036003 | positive regulation of transcription from RNA | 22 | 2.00E−04 |
| | | | polymerase II promoter in response to stress | | |
| ncma processing biogenesis | Translation | GO:0042254 | ribosome biogenesis | 279 | 1.62E−09 |
| topologically incorrect protein | Response to stimulus | GO:0034976 | response to endoplasmic reticulum stress | 253 | 3.04E−07 |
| er nucleus signaling | Response to stimulus | GO:0036499 | PERK-mediated unfolded protein response | 20 | 5.55E−06 |
| positive regulation creb | Signal transduction | GO:0032793 | positive regulation of CREB transcription factor | 12 | 2.27E−04 |
| | | | activity | | |
| cyclic nucleotide process | Signal transduction | GO:0046058 | CAMP metabolic process | 14 | 4.80E−03 |
| utilizing autophagic mechanism | Autophagy | GO:0006914 | autophagy | 452 | 1.90E−03 |
| selective organell disassembly | Autophagy | GO:0000422 | autophagy of mitochondrion | 70 | 4.94E−03 |
| sphingolipid biosynthetic | Metabolism | GO:0090154 | positive regulation of sphingolipid biosynthetic | 10 | 3.94E−03 |
| ceramide | | | process | | |
| pentose phosphate shunt | Metabolism | GO:0006098 | pentose phosphate shunt | 14 | 6.44E−03 |
| Downregulated | | | | | |
| exogenous peptide antigen | Antigen presentation | GO:0019882 | antigen processing and presentation | 207 | 1.03E−08 |
| organization actin filament | Cytoskeleton | GO:0032956 | regulation of actin cytoskeleton organization | 262 | 6.57E−06 |
| | organization | | | | |
| Lipid catabolic glycolipid | Metabolism | GO:0046466 | membrane lipid catabolic process | 28 | 3.74E−03 |

TABLE 3
Pathway enrichment analysis of DCATH-2 primed LPS-stimulated dTHP-1 cells.

| | | | | Gene | |
|---|---|---|---|---|---|
| | | Gene | | set | FDR |
| Cluster | Biological theme | ontology | GO biological process | size | Q-value |
| Upregulated | | | | | |
| protein targeting er | Translation | GO:0000184 | nuclear-transcribed mRNA catabolic process, | 119 | 4.04E−05 |
| | | | nonsense-mediated decay | | |
| protein targeting er | Translation | GO:0006413 | translational initiation | 184 | 4.04E−05 |
| interleukin 1 mediated | Signal transduction | GO:0070498 | interleukin-1 mediated signaling pathway | 95 | 8.56E−04 |
| tyrosine phosphorylation stat | Signal transduction | GO:0042531 | (+) regulation of Tyr phosphorylation of STAT | 44 | 4.28E−03 |
| | | | protein | | |
| molecule bacterial | Immune response | GO:0071222 | cellular response to lipopolysaccharide | 165 | 8.14E−05 |
| lipopolysaccharide | | | | | |
| Chemokine cytokine biosynthetic | Immune response | GO:0045073 | regulation of chemokine biosynthetic process | 13 | 1.29E−03 |
| prostanoid process prostaglandin | Immune response | GO:0001516 | prostaglandin biosynthetic process | 19 | 8.84E−03 |
| macrophage foam differentiation | Differentiation | GO:0030730 | sequestering of triglyceride | 11 | 9.52E−04 |
| pentose phosphate shunt | Metabolism | GO:0006098 | pentose-phosphate shunt | 14 | 3.10E−03 |
| sphingolipid biosynthetic ceramide | Metabolism | GO:0090154 | positive regulation of sphingolipid biosynthetic | 10 | 7.44E−03 |
| | | | process | | |
| Downregulated | | | | | |
| exogenous peptide antigen | Antigen presentation | GO:0002495 | antigen processing and presentation of peptide or | 89 | 1.41E−04 |
| | | | polysaccharide antigen via MHC class II | | |

### REFERENCES

1. Ifrim, D. C., et al., Trained immunity or tolerance: opposing functional programs induced in human monocytes after engagement of various pattern recognition receptors. Clin Vaccine Immunol, 2014. 21(4): p. 534-45.
2. Quintin, J., et al., Candida albicans infection affords protection against reinfection via functional reprogramming of monocytes. Cell Host Microbe, 2012. 12(2): p. 223-32.
3. Kleinnijenhuis, J., et al., Bacille Calmette-Guerin induces NOD2-dependent nonspecific protection from reinfection via epigenetic reprogramming of monocytes. Proc Natl Acad Sci USA, 2012. 109(43): p. 17537-42.
4. Kleinnijenhuis, J., et al., BCG-induced trained immunity in NK cells: Role for non-specific protection to infection. Clin Immunol, 2014. 155(2): p. 213-9.
5. van der Valk, F. M., et al., Oxidized Phospholipids on Lipoprotein(a) Elicit Arterial Wall Inflammation and an Inflammatory Monocyte Response in Humans. Circulation, 2016. 134(8): p. 611-24.
6. Cheng, S.C., et al., mTOR- and HIF-1alpha-mediated aerobic glycolysis as metabolic basis for trained immunity. Science, 2014. 345(6204): p. 1250684.
7. Scheenstra, M. R., et al., Cathelicidins Modulate TLR-Activation and Inflammation. Front Immunol, 2020. 11: p. 1137.
8. Yang, D., et al., Alarmin-induced cell migration. Eur J Immunol, 2013. 43(6): p. 1412-8.
9. Coorens, M., et al., Interspecies cathelicidin comparison reveals divergence in antimicrobial activity, TLR modulation, chemokine induction and regulation of phagocytosis. Sci Rep, 2017. 7: p. 40874.
10. van Dijk, A., et al., Identification of chicken cathelicidin-2 core elements involved in antibacterial and immunomodulatory activities. Mol Immunol, 2009. 46(13): p. 2465-73.
11. van Dijk, A., et al., Immunomodulatory and Anti-Inflammatory Activities of Chicken Cathelicidin-2 Derived Peptides. PLoS One, 2016. 11(2): p. e0147919.
12. Mookherjee, N., et al., Antimicrobial host defence peptides: functions and clinical potential. Nat Rev Drug Discov, 2020. 19(5): p. 311-332.
13. Nijnik, A., et al., Synthetic cationic peptide IDR-1002 provides protection against bacterial infections through chemokine induction and enhanced leukocyte recruitment. J Immunol, 2010. 184(5): p. 2539-50.
14. Rivas-Santiago, B., et al., Ability of innate defence regulator peptides IDR-1002, IDR-HH2 and IDR-1018 to protect against Mycobacterium tuberculosis infections in animal models. PLoS One, 2013. 8(3): p. e59119.
15. Bommineni, Y. R., et al., A fowlicidin-1 analog protects mice from lethal infections induced by methicillin-resistant Staphylococcus aureus. Peptides, 2010. 31(7): p. 1225-30.
16. Garton, M., et al., Method to generate highly stable D-amino acid analogs of bioactive helical peptides using a mirror image of the entire PDB. Proc Natl Acad Sci USA, 2018. 115(7): p. 1505-1510.
17. Cuperus, T., et al., Protective effect of in ovo treatment with the chicken cathelicidin analog D-CATH-2 against avian pathogenic E. coli. Sci Rep, 2016. 6: p. 26622.
18. Schneider, V. A., et al., Prophylactic administration of chicken cathelicidin-2 boosts zebrafish embryonic innate immunity. Dev Comp Immunol, 2016. 60: p. 108-14.
19. Cuperus, T., et al., Immunomodulation and effects on microbiota after in ovo administration of chicken cathelicidin-2. PLoS One, 2018. 13(6): p. e0198188.
20. Bekkering, S., et al., Oxidized low-density lipoprotein induces long-term proinflammatory cytokine production and foam cell formation via epigenetic reprogramming of monocytes. Arterioscler Thromb Vasc Biol, 2014. 34(8): p. 1731-8.
21. Park, E. K., et al., Optimized THP-1 differentiation is required for the detection of responses to weak stimuli. Inflamm Res, 2007. 56(1): p. 45-50.
22. Behnsen, J., et al., Exploiting host immunity: the Salmonella paradigm. Trends Immunol, 2015. 36(2): p. 112-20.
23. Wellington, M., K. Dolan, and D. J. Krysan, Live Candida albicans suppresses production of reactive oxygen species in phagocytes. Infect Immun, 2009. 77(1): p. 405-13.
24. Wagener, J., et al., Candida albicans Chitin Increases Arginase-1 Activity in Human Macrophages, with an Impact on Macrophage Antimicrobial Functions. MBio, 2017. 8(1).
25. Dibble, C. C. and B. D. Manning, Signal integration by mTORC1 coordinates nutrient input with biosynthetic output. Nat Cell Biol, 2013. 15(6): p. 555-64.
26. Arts, R. J. W., et al., BCG Vaccination Protects against Experimental Viral Infection in Humans through the Induction of Cytokines Associated with Trained Immunity. Cell Host Microbe, 2018. 23(1): p. 89-100 e5.
27. Olson, C. M., et al., p38 mitogen-activated protein kinase controls NF-kappaB transcriptional activation and tumor necrosis factor alpha production through RelA phosphorylation mediated by mitogen- and stress-activated protein kinase 1 in response to Borrelia burgdorferi antigens. Infect Immun, 2007. 75(1): p. 270-7.
28. Thakur, V., et al., Chronic ethanol feeding increases activation of NADPH oxidase by lipopolysaccharide in rat Kupffer cells: role of increased reactive oxygen in LPS-stimulated ERK1/2 activation and TNF-alpha production. J Leukoc Biol, 2006. 79(6): p. 1348-56.
29. Goral, J., M. A. Choudhry, and E. J. Kovacs, Acute ethanol exposure inhibits macrophage IL-6 production: role of p38 and ERK1/2 MAPK. J Leukoc Biol, 2004. 75(3): p. 553-9.
30. Tang, X., et al., P2X7 Receptor Regulates Internalization of Antimicrobial Peptide LL-37 by Human Macrophages That Promotes Intracellular Pathogen Clearance. J Immunol, 2015. 195(3): p. 1191-201.
31. Tomasinsig, L., et al., The human cathelicidin LL-37 modulates the activities of the P2X7 receptor in a structure-dependent manner. J Biol Chem, 2008. 283(45): p. 30471-81.
32. Raudvere, U., et al., g:Profiler: a web server for functional enrichment analysis and conversions of gene lists (2019 update). Nucleic Acids Res, 2019. 47(W1): p. W191→W198.
33. Shannon, P., et al., Cytoscape: a software environment for integrated models of biomolecular interaction networks. Genome Res, 2003. 13(11): p. 2498-504.
34. Merico, D., et al., Enrichment map: a network-based method for gene-set enrichment visualization and interpretation. PLoS One, 2010. 5(11): p. e13984.

1. (canceled)

2. (canceled)

3. (canceled)

4. A method for activating, inducing or promoting an innate immune memory in a subject in need thereof, the method comprising administering to said subject an effective amount of CATH2 or a derivative thereof, thereby activating, inducing or promoting innate immune memory in said subject.

5. The method according to claim 4 wherein the innate immune memory is innate immune memory for infectious disease.

6. The method according to claim 4 for treatment or prevention of infectious disease.

7. The method according to claim 4 for improving or enhancing antimicrobial activity of an antimicrobial agent.

8. (canceled)

9. A method for improving or enhancing antimicrobial activity of an antimicrobial agent comprising administering to a subject in need thereof an effective amount of CATH2 or a derivative thereof, thereby improving or enhancing antimicrobial activity.

10. The method according to claim 4, wherein the treatment is treatment of Salmonella enteritidis or Candida albicans.

11. The method according to claim 4 wherein the CATH2 or derivative thereof is administered before, after or simultaneously with a treatment with a pathogenic microorganism or an antigenic part thereof.

12. The method according to claim 4 wherein the subject in need thereof is suffering from an infectious disease or at risk of suffering from an infectious disease, or the subject is in contact with subjects suffering from said infectious disease.

13. The method according to claim 4 comprising administering said CATH2 or derivative thereof to subjects of a population of subjects wherein an infectious disease has been established in one or more subjects of said population.

14. The method according to claim 4 wherein the CATH2 or a derivative thereof is combined with an adjuvant specific for innate immunity, such as a toll-like receptor (TLR) ligands, β-glucan, muramyl dipeptide (MDP), Bacille Calmette-Guerin (BCG), CpG oligodeoxynucleotide.

15. The method according to claim 4 wherein the subject is livestock, a farm animal, a pet, or poultry.

16. The method according to claim 4 wherein the subject is poultry and the administration is performed in ovo and/or after hatch.

17. The method according to claim 4 wherein the subject is administered the CATH2 derivative twice, preferably with an interval of at least 2 days.

18. The method according to according to claim 17, wherein the subject is poultry and one of the administrations is in ovo administration and one of the administrations is administration after hatch.

19. (canceled)

20. A method for the treatment or prevention of an infectious disease caused by a pathogen, comprising administering a pathogen-specific vaccine that is specific for said pathogen and CATH2 or a derivative thereof as an adjuvant, preferably wherein the pathogen-specific vaccine is an (attenuated) pathogen or pathogen derived peptide or protein.

21. The method according to claim 4 wherein the CATH2 derivative is selected from the group consisting of DCATH2, a C-terminally and/or N-terminally truncated CATH2 and a C-terminally or N-terminally truncated DCATH2.

22. The method according to claim 4 for treating an inactivated or defective innate immune memory in the subject.

23. The method according to claim 7, wherein the antimicrobial agent is the CATH2 or derivative thereof.

24. The method according to claim 21, wherein the CATH2 derivative is selected from the group consisting of DCATH-2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

25. The method according to claim 21, wherein the CATH2 or derivative is DCATH2, DCATH2(1-21) or DCATH2(4-21).


SEQUENCE LISTING (28 sequences)

SEQ ID NO: 1 - protein, 27 aa, Artificial Sequence
  C-terminal peptide called CMAP27 or CATH2
RFGRFLRKIRRFRPKVTITIQGSARFG

SEQ ID NO: 2 - protein, 26 aa, Artificial Sequence
  D-amino acid CATH2 derivative D-C(1-26)
  MISC_FEATURE (1)..(26): D-amino acid
  MOD_RES (26)..(26): AMIDATION
RFGRFLRKIRRFRPKVTITIQGSARF

SEQ ID NO: 3 - protein, 21 aa, Artificial Sequence
  D-amino acid CATH2 derivative D-C(1-21)
  MISC_FEATURE (1)..(21): D-amino acid
  MOD_RES (21)..(21): AMIDATION
RFGRFLRKIRRFRPKVTITIQ

SEQ ID NO: 4 - protein, 18 aa, Artificial Sequence
  D-amino acid CATH2 derivative D-C(4-21)
  MISC_FEATURE (1)..(18): D-amino acid
  MOD_RES (18)..(18): AMIDATION
RFLRKIRRFRPKVTITIQ

SEQ ID NO: 5 - protein, 15 aa, Artificial Sequence
  D-amino acid CATH2 derivative D-C(7-21)
  MISC_FEATURE (1)..(15): D-amino acid
  MOD_RES (15)..(15): AMIDATION
RKIRRFRPKVTITIQ

SEQ ID NO: 6 - protein, 15 aa, Artificial Sequence
  D-amino acid CATH2 derivative D-C(7-21) F/W
  MISC_FEATURE (1)..(15): D-amino acid
  MOD_RES (15)..(15): AMIDATION
RKIRRWRPKVTITIQ

SEQ ID NO: 7 - protein, 15 aa, Artificial Sequence
  D-amino acid CATH2 derivative D-C(7-21) F/Y
  MISC_FEATURE (1)..(15): D-amino acid
  MOD_RES (15)..(15): AMIDATION
RKIRRYRPKVTITIQ

SEQ ID NO: 8 - protein, 12 aa, Artificial Sequence
  D-amino acid CATH2 derivative D-C(10-21) F/W
  MISC_FEATURE (1)..(12): D-amino acid
  MOD_RES (12)..(12): AMIDATION
RRWRPKVTITIQ

SEQ ID NO: 9 - protein, 15 aa, Artificial Sequence
  D-amino acid CATH2 derivative D-C (1-15)
  MISC_FEATURE (1)..(15): D-amino acid
RFGRFLRKIRRFRPK

SEQ ID NO: 10 - protein, 21 aa, Artificial Sequence
  cycCMAP(1-21)[Lys8]
  MISC_FEATURE (1)..(21): cyclic derivative
  MISC_FEATURE (8)..(8): is Fmoc-L-bishomopropargylglycine
  MISC_FEATURE (21)..(21): azido resin attached
RFGRFLRXIRRFRPKVTITIQ

SEQ ID NO: 11 - protein, 21 aa, Artificial Sequence
  cycCMAP(1-21)[Arg7]
  MISC_FEATURE (1)..(21): cyclic derivative
  MISC_FEATURE (7)..(7): is Fmoc-L-bishomopropargylglycine
  MISC_FEATURE (21)..(21): azido resin attached
RFGRFLXKIRRFRPKVTITIQ

SEQ ID NO: 12 - protein, 21 aa, Artificial Sequence
  cycCMAP(1-21)[Leu6]
  MISC_FEATURE (1)..(12): cyclic derivative
  MISC_FEATURE (6)..(6): is Fmoc-L-bishomopropargylglycine
  MISC_FEATURE (21)..(21): azido resin attached
RFGRFXRKIRRFRPKVTITIQ

SEQ ID NO: 13 - protein, 21 aa, Artificial Sequence
  cycCMAP(1-21)[Leu6],Phe2/Trp
  MISC_FEATURE (1)..(21): cyclic derivative
  MISC_FEATURE (6)..(6): is Fmoc-L-bishomopropargylglycine
  MISC_FEATURE (21)..(21): azido resin attached
RWGRFXRKIRRFRPKVTITIQ

SEQ ID NO: 14 - protein, 21 aa, Artificial Sequence
  cycCMAP(1-21)[Leu6],Phe2,5/Trp
  MISC_FEATURE (1)..(21): cyclic derivative
  MISC_FEATURE (6)..(6): is Fmoc-L-bishomopropargylglycine
  MISC_FEATURE (21)..(21): azido resin attached
RWGRWXRKIRRFRPKVTITIQ

SEQ ID NO: 15 - protein, 21 aa, Artificial Sequence
  cycCMAP(1-21)[Leu6],Phe2,5,12/Trp
  MISC_FEATURE (1)..(21): cyclic derivative
  MISC_FEATURE (6)..(6): is Fmoc-L-bishomopropargylglycine
  MISC_FEATURE (21)..(21): azido resin attached
RWGRWXRKIRRWRPKVTITIQ

SEQ ID NO: 16 - protein, 21 aa, Artificial Sequence
  cycCMAP(1-21)[Leu6],Phe5,12/Trp
  MISC_FEATURE (1)..(21): cyclic derivative
  MISC_FEATURE (6)..(6): is Fmoc-L-bishomopropargylglycine
  MISC_FEATURE (21)..(21): azido resin attached
RFGRWXRKIRRWRPKVTITIQ

SEQ ID NO: 17 - protein, 21 aa, Artificial Sequence
  cycCMAP(1-21)[Leu6],Phe12/Trp
  MISC_FEATURE (1)..(21): cyclic derivative
  MISC_FEATURE (6)..(6): is Fmoc-L-bishomopropargylglycine
  MISC_FEATURE (21)..(21): azido resin attached
RFGRFXRKIRRWRPKVTITIQ

SEQ ID NO: 18 - protein, 27 aa, Artificial Sequence
  I and RI equivalent of CATH2
GFRASGQITITVKPRFRRIKRLFRGFR

SEQ ID NO: 19 - protein, 21 aa, Artificial Sequence
  RI-CMAP27-derivative containing one or more D amino acids RI-C(1-21)
QITITVKPRFRRIKRLFRGFR

SEQ ID NO: 20 - protein, 18 aa, Artificial Sequence
  RI-CMAP27-derivative containing one or more D amino acids RI-C(4-21)
QITITVKPRFRRIKRLFR

SEQ ID NO: 21 - protein, 15 aa, Artificial Sequence
  RI-CMAP27-derivative containing one or more D amino acids RI-C(7-21)
QITITVKPRFRRIKR

SEQ ID NO: 22 - protein, 15 aa, Artificial Sequence
  RI-CMAP27-derivative containing one or more D amino acids RI-C(7-21)F/W
QITITVKPRWRRIKR

SEQ ID NO: 23 - protein, 15 aa, Artificial Sequence
  RI-CMAP27-derivative containing one or more D amino acids RI-C(7-21)F/Y
QITITVKPRYRRIKR

SEQ ID NO: 24 - protein, 12 aa, Artificial Sequence
  RI-CMAP27-derivative containing one or more D amino acids RI-C(10-21)F/W
QITITVKPRWRR

SEQ ID NO: 25 - protein, 26 aa, Gallus gallus
  MOD_RES (26)..(26): AMIDATION
RFGRFLRKIRRFRPKVTITIQGSARF

SEQ ID NO: 26 - protein, 26 aa, Artificial Sequence
  D-amino acid analog, DCATH-2
  MISC_FEATURE (1)..(26): D-amino acid
  MOD_RES (26)..(26): AMIDATION
RFGRFLRKIRRFRPKVTITIQGSARF

SEQ ID NO: 27 - protein, 37 aa, Homo sapiens
LLGDFFRKSKEKIGKEFKRIVQRIKDFLRNLVPRTES

SEQ ID NO: 28 - protein, 26 aa, Artificial Sequence
  Tamra-labeled DCATH-2
  MISC_FEATURE (1)..(1): Tamra-labeled
  MISC_FEATURE (1)..(26): D-amino acid
  MOD_RES (26)..(26): AMIDATION
RFGRFLRKIRRFRPKVTITIQGSARF